(19) United States
(12) Patent Application Publication
(10) Pub. No.: US 2022/0062390 A1
(43) Pub. Date: Mar. 3, 2022

(54) METHODS OF TREATING CANCER

(71) Applicant: Dr. Reddy's Laboratories SA, Basel (CH)

(72) Inventors: Anil Namboodiripad, Yardley, PA (US); A. Raghav Chari, New York, NY (US); Chandrasekhar Goda, Belle Mead, NJ (US); Mary Woodall-Jappe, Ipswich, MA (US); Preeti Singh, Yardley, PA (US)

(73) Assignee: Dr. Reddy's Laboratories SA, Basel (CH)

(21) Appl. No.: 17/409,589

(22) Filed: Aug. 23, 2021

Related U.S. Application Data
(60) Provisional application No. 63/070,645, filed on Aug. 26, 2020.

(51) Int. Cl. A61K 38/45; A61K 38/20; A61K 39/395; A61P 35/00

(52) U.S. Cl. CPC A61K 38/45; A61P 35/00; A61K 39/3955; A61K 38/2013

(57) ABSTRACT

One aspect of the technology described herein relates to a method for treating a subject having a cancer. This method involves selecting a subject having cancer; administering, to the selected subject, a cytotoxic fusion protein comprising an N-terminus coupled to a C-terminus, wherein the N-terminus comprises diphtheria toxin fragments A and B and the C-terminus comprises human IL-2; and administering, to the selected subject, a programmed cell death-1 receptor (PD-1) pathway inhibitor to treat the cancer in the subject. Also disclosed is a method for sensitizing a target cell population to treatment with a PD-1 pathway inhibitor, as well as compositions and kits for use in treating a subject having cancer.

This application claims the priority benefit of U.S. Provisional Patent Application Ser. No. 63/070,645, filed Aug. 26, 2020, which is hereby incorporated by reference in its entirety.

### FIELD

Certain aspects of the technology disclosed herein relate to methods for treating a subject having cancer. Also disclosed in certain embodiments are methods for sensitizing a target cell population to treatment with a PD-1 inhibitor. Other embodiments are directed to compositions and kits for use in treating a subject having cancer.

### BACKGROUND

Cutaneous T-cell lymphoma (CTCL) is a form of non-Hodgkin lymphoma, caused by the mutation of T-cells. Malignant T cells in the body migrate to the skin, causing various lesions to appear. As the disease progresses, the rash that appears eventually forms plaques and tumors before metastasizing to other body parts. Diagnosis is difficult early in the course of this disease because it mimics several benign skin disorders, including eczema, psoriasis, and contact dermatitis.

Currently, there is no cure for CTCL. ONTAK® (denileukin diftitox) is a recombinant cytotoxic fusion protein, composed of the amino acid sequences for diphtheria toxin fragments A and B and interleukin-2, indicated for the treatment of patients with persistent or recurrent cutaneous T-cell lymphoma whose malignant cells express the CD25 component of the IL-2 receptor. ONTAK® has been used to target CD25⁺ lymphoma cells, as well as T regulatory (Treg) cells, and activated T effector (Teff) cells in syndromes ranging from stage IV unresectable malignant melanoma to steroid-resistant graft-versus-host disease (Lansigan et al., “Role of Denileukin diftitox in the Treatment of Persistent or Recurrent Cutaneous T-cell Lymphoma,” Cancer Manag. Res. 2:53-59 (2010); Telang et al., “Phase II Trial of the Regulatory T Cell-Depleting Agent, Denileukin diftitox, in Patients with Unresectable Stage IV Melanoma,” BMC Cancer 11:515 (2011); and Ho et at, “Safety and Efficacy of Denileukin diftitox in Patients with Steroid-Refractory Acute Graft-Versus-Host Disease After Allogeneic Hematopoietic Stem Cell Transplantation,” Blood 104:1224-1226 (2004)).

Programmed death protein 1 (PD-1) is an inhibitory receptor expressed by activated B cells, T cells, and natural killer (NK) cells, as well as some myeloid cells. PD-1 and its ligands, PD-L1 and PD-L2, control immune activity by causing a transient downregulation of T-cell function. Upregulated expression of PD-L1 on tumor and/or stromal cells in the tumor microenvironment enables engagement of PD-1 on activated T cells and functions to down-regulate T-cell activation, resulting in diminished antitumor T-cell responses.

The fully human immunoglobulin G4 (IgG4) monoclonal antibody nivolumab (OPDIVO®, Bristol-Myers Squibb) and the humanized IgG4-κ monoclonal antibody pembrolizumab (KEYTRUDA®, Merck) target PD-1 to reverse the inhibitory signal and increase antitumor activity. Similarly, monoclonal antibodies have been developed against PD-L1, including the humanized IgG1 agent atezolizumab (TECENTRIQ®, Genentech) and the fully human IgG1 agents avelumab (BAVENCIO®, EMD Serono/Pfizer) and durvalumab (IMFINZI®, AstraZeneca).

There is a need for additional immunomodulatory compositions and methods suitable for the treatment of cancer (e.g., CTCL) and other T-cell mediated diseases.

The present invention is directed to overcoming these and other deficiencies in the art.

### SUMMARY

One aspect of the technology disclosed herein relates to a method for treating a subject having a cancer. This method involves administering to the subject a composition comprising a monomeric cytotoxic fusion protein comprising an N-terminus coupled to a C terminus, where the N-terminus comprises diphtheria toxin fragments A and B and the C-terminus comprises human IL-2, and where at least 95.0% of the total cytotoxic fusion protein content of the composition is a monomeric cytotoxic fusion protein; and administering to the subject a programmed cell death-1 receptor (PD-1) pathway inhibitor to treat cancer in the subject. The combination of agents can be simultaneous or sequential as long as there is an overlap in the plasma concentration of the two agents. In some embodiments, the human IL-2 is full-length human IL-2.

Another aspect of the technology described herein relates to a method of sensitizing a target cell population to treatment with a PD-1 pathway inhibitor. This method involves selecting a target cell population and administering to the selected target cell population (e.g., to an individual patient or patient population) a composition comprising a monomeric cytotoxic fusion protein comprising an N-terminus coupled to a C-terminus, where the N-terminus comprises diphtheria toxin fragments A and B and the C-terminus comprises human IL-2, where at least 95.0% of the total cytotoxic fusion protein content of the composition is a monomeric cytotoxic fusion protein, and where said administering is effective to sensitize the target cell population to treatment with the PD-1 pathway inhibitor. This method also encompasses optionally administering a PD-1 pathway inhibitor (e.g., after the sensitization) to the selected target cell population (e.g., to an individual patient or patient population). In some embodiments, the human IL-2 is full-length human IL-2.

Another aspect is directed to compositions and kits for use in treating a subject having cancer, the composition or kit comprising (i) a monomeric cytotoxic fusion protein comprising an N-terminus coupled to a C-terminus, where the N-terminus comprises diphtheria toxin fragments A and B and the binding domain at the C-terminus comprises human IL-2, and where at least 95.0% of the total cytotoxic fusion protein content of the composition is a monomeric cytotoxic fusion protein; and (ii) a programmed cell death-1 receptor (PD-1) pathway inhibitor. In some embodiments, the human IL-2 is full-length human IL-2.

### BRIEF DESCRIPTION OF THE DRAWINGS

FIG. 1 is a schematic illustration of a cytotoxic fusion protein comprising an N terminus coupled to a C-terminus, where the N-terminus comprises amino acid sequences of diphtheria toxin fragments A and B, and the C-terminus comprises the amino acid sequence of human interleukin-2 (IL-2). The N-terminus of the cytotoxic fusion protein mediates binding to the IL-2 receptor on T-cell lymphoma cells.

FIG. 2 is a schematic illustration showing IL-2 receptor (IL-2R) binding and internalization of the cytotoxic fusion protein on the surface of cells expressing the high-affinity human IL-2R. The high-affinity human IL-2R comprises three membrane proteins: the 55 kD IL-2Rα chain (TAC, CD25), the 70-75 kD IL-2Rβ chain (CD122), and the 64 kD IL-2Rγ chain (CD132). The N-terminus of the cytotoxic fusion protein interacts with the high (CD25/CD122/CD132) affinity IL-2 receptors on the surface of a target cell and undergoes internalization by receptor-mediated endocytosis. Endosome acidification and furin-protease mediated toxin cleavage in the endosome release the diphtheria toxin fragment A portion of the cytotoxic fusion protein into the cytosol, where it inhibits cellular protein synthesis and results in rapid cell death (Erter et al., “New Targets of Therapy in T-Cell Lymphomas,” Curr. Drug Targets 11(14): 482-493 (2010), which is hereby incorporated by reference in its entirety).

FIGS. 3A-3D demonstrate the therapeutic efficacy of various treatments on tumor growth inhibition in three murine models of cancer. FIG. 3A outlines the study design of experiments used to evaluate the efficacy of combined administration of a cytotoxic fusion protein in combination with a PD-1 inhibitor in an H22 murine liver syngeneic model in female BALB/c mice, a CT26 murine colon syngeneic model in female BALB/c mice, and a B16F10 murine melanoma syngeneic model in female C57BL/6 mice. Each study began with 24 mice per group. Briefly, tumors were implanted subcutaneously into mice. Next, mice were randomized into groups with equal-sized tumors and treated with: vehicle control (Group 1); cytotoxic fusion protein monotherapy (Group 2); anti-PD-1 monotherapy (Group 3); cytotoxic fusion protein+anti-PD-1, concurrent administration (Group 4); cytotoxic fusion protein 2 days prior to anti-PD-1 (Group 5); or anti-PD-1 2 days prior to cytotoxic fusion protein (Group 6). 12 of the mice in each group were culled at various time points and used as tissue sources for biomarker exploration. 4 mice were culled 24 hours post 1^{st} dose (day 1 for Group 1, Group 2, Group 3, and Group 4; day 3 for Group 5 and Group 6)—if the tumor size<200 mm³, tumors from 2 mice were pooled together; tumor-draining lymph nodes were harvested for fluorescence activated cell sorting (FACS); spleens were harvested and halved for FACS and immunohisto-chemistry (IHC). 4 mice were culled 24 hours post 1^{st} dose (day 1 for Group 1, Group 2, Group 3, and Group 4; day 3 for Group 5 and Group 6)—tumor and tumor-draining lymph nodes were harvested for IHC. 4 mice were culled at day 8 for Group 1 and Group 2; day 9 for Group 3 and Group 4; day 11 for Group 5 and Group 6—tumor-draining lymph nodes were harvested from 2 mice for FACS; tumor-draining lymph nodes were harvested from 2 mice for IHC; half of the spleen and tumor was harvested for FACS and the other half of the spleen and tumor was harvested for IHC. Tumor draining lymph nodes (TDLN) were harvested at termination on Day 11 (B16F10 melanoma), Day 14 (CT26 colon), or Day 23 (H22 liver). FIG. 3B shows the mean tumor volume (top panel) and mean body weight (bottom panel) in mice evaluated using the H22 liver carcinoma model. FIG. 3C shows the mean tumor volume (top panel) and mean body weight (bottom panel) in mice evaluated using the CT26 colon carcinoma model. FIG. 3D shows the mean tumor volume (top panel) and mean body weight (bottom panel) in mice evaluated using the B16F10 melanoma model.

FIGS. 4A-4F are graphs showing the percentage of CD8⁺ and FoxP3⁺ cells within tumors (FIGS. 4A and 4D, respectively), spleens (FIGS. 4B and 4E, respectively), and tumor-draining lymph nodes (FIGS. 4C and 4F, respectively) present in mice within Group 1, Group 2, Group 3, Group 4, Group 5, and Group 6 from the H22 liver cancer syngeneic model at various time points. Tumor, spleen, and tumor-draining lymph node tissue samples from BALB/c mice implanted with H22 tumor cells were collected on day 1 for Group 1, Group 2, Group 3, and Group 4 (1^{st} collection); day 3 for Group 5 and Group 6 (1^{st} collection); day 8 for Group 1 and Group 2 (2^{nd} collection); day 9 for Group 3 and Group 4 (2^{nd} collection); and day 11 for Group 5 and Group 6 (2^{nd} collection). Tumors were also collected on day 23 when all remaining mice were euthanized.

FIGS. 5A-5B are images showing immunohistochemical staining of CD8 (FIG. 5A) and FoxP3 (FIG. 5B) tumor, spleen, and tumor-draining lymph node tissue samples from Group 1, Group 2, Group 3, Group 4, Group 5, and Group 6 from the H22 liver cancer syngeneic model collected at various time points. Tumor, spleen, and tumor draining lymph node tissue samples from BALB/c mice implanted with H22 tumor cells were collected on day 1 for Group 1, Group 2, Group 3, and Group 4 (1^{st} collection); day 3 for Group 5 and Group 6 (1^{st} collection); day 8 for Group 1 and Group 2 (2^{nd} collection); day 9 for Group 3 and Group 4 (2^{nd} collection); and day 11 for Group 5 and Group 6 (2^{nd} collection). Tumors were also collected on day 23 when all remaining mice were euthanized.

FIGS. 6A-6D demonstrates the effect of various treatments on tumor growth inhibition and the survival of female BALB/c mice bearing H22 murine liver tumors. The study began with 16 mice per group. Briefly, tumors were implanted subcutaneously into mice. Next, mice were randomized into groups with equal-sized tumors and treated with: vehicle control (Group 1); cytotoxic fusion protein monotherapy was administered once per 7 days for 3 treatments (Q7Dx3) (Group 2); anti-PD-1 monotherapy was administered once per 4 days for 6 treatments (Q4Dx6) (Group 3); cytotoxic fusion protein+anti-PD-1, with the initial administration of both treatments started on the same day (“concurrent” group) (Group 4); an initial dose of cytotoxic fusion protein administered 2 days prior to the initial dose of anti-PD-1 (Group 5). All treatments were completed by day 22. Mice were observed daily, with body weights and tumor volumes measured every three days throughout the duration of the study. FIGS. 6A-6C show the mean tumor volume (FIG. 6A), the percent inhibition of tumor volume (FIG. 6B), and mean body weight (FIG. 6C) in mice evaluated using the H22 liver carcinoma model. The observation period continued for the subsequent 78 days after dosing was completed, or until each animal succumbed to the tumor or was euthanized according to Institutional Animal Care and Use Committee (IACUC) guidelines for humane care. FIG. 6D is a Kaplan-Meier curve showing survival analysis of mice in Groups 1-5 on Day 73.

FIGS. 7A-7D demonstrates the effect of various treatments on tumor growth inhibition and the survival rate of female BALB/c mice bearing C26 murine colon tumors. Each study began with 16 mice per group. Briefly, tumors were implanted subcutaneously into mice. Next, mice were randomized into groups with equal-sized tumors and treated with: vehicle control (Group 1); cytotoxic fusion protein monotherapy was administered once per 7 days for 3 treatments (Q7Dx3) (Group 2); anti-PD-1 monotherapy was administered once per 4 days for 6 treatments (Q4Dx6) (Group 3); cytotoxic fusion protein+anti-PD-1, with the initial administration of both treatments started on the same day (“concurrent” group) (Group 4); an initial dose of cytotoxic fusion protein administered 2 days prior to the initial dose of anti-PD-1 (Group 5). All treatments were completed by day 22. Mice were observed daily, with body weights and tumor volumes measured every three days throughout the duration of the study. FIGS. 7A-7C show the mean tumor volume (FIG. 7A), the percent inhibition of tumor volume (FIG. 7B), and mean body weight (FIG. 7C) in mice evaluated using the CT26 colon carcinoma model. The observation period continued for the subsequent 78 days after dosing was completed, or until each animal succumbed to the tumor or was euthanized according to IACUC guidelines for humane care. FIG. 7D is a Kaplan-Meier curve showing survival analysis of mice in Groups 1-5 on Day 73.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The terms “comprising,” “comprises,” and “comprised of” also encompass the term “consisting of.” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

Terms of degree such as “substantially,” “about,” and “approximately” as used herein mean a reasonable amount of deviation of the modified term such that the end result is not significantly changed. These terms of degree should be construed as including a deviation of at least ±1% (and up to ±5% or ±10%) of the modified term if this deviation would not negate the meaning of the word it modifies.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints.

As used herein, “synergy” or “synergistic effect” with regard to an effect produced by two or more individual components refers to a phenomenon in which the total effect produced by these components, when utilized in combination, is greater than the sum of the individual effects of each component acting alone.

The terms “isolated” or “purified” refer to material that is substantially or essentially free from components that normally accompany it as found in its native state. Purity and homogeneity are typically determined using analytical chemistry techniques such as polyacrylamide gel electrophoresis or high-performance liquid chromatography. A protein that is the predominant species present in a preparation is substantially purified.

The term “patient” means a subject (preferably a human) who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient” and inclusive of the term “healthy subject” (i.e., an individual (e.g., a human) who is entirely normal in all respects or with respect to a particular condition.

The terms “treatment of” and “treating” include the administration of an active agent(s) with the intent to lessen the severity of a condition.

The terms “prevention of” and “preventing” include the avoidance of the onset of a condition by a prophylactic administration of the active agent. One aspect of the technology described herein relates to a method for treating a subject having a cancer. This method involves administering to the subject a composition comprising a monomeric cytotoxic fusion protein comprising an N-terminus coupled to a C-terminus, where the N-terminus comprises diphtheria toxin fragments A and B and the C-terminus comprises human IL-2, and where at least 95.0% of the total cytotoxic fusion protein content of the composition is a monomeric cytotoxic fusion protein; and administering to the subject a programmed cell death-1 receptor (PD-1) pathway inhibitor to treat cancer in the subject. The human IL-2 may comprise a receptor-binding domain of human IL-2. In some embodiments, the human IL-2 is full-length human IL-2.

Suitable subjects in accordance with the methods described herein include, without limitation, a mammal, e.g., a human. In certain embodiments, the subject is an infant, a child, an adolescent, a young adult, an adult, or a geriatric adult. Additional suitable subjects include, but are not limited to, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In some embodiments, the subject has been previously treated with a cytotoxic fusion protein monotherapy. In some embodiments, the subject has been previously treated with a PD-1 inhibitor monotherapy. In other embodiments, the subject has not been previously treated with a cytotoxic fusion protein monotherapy. In other embodiments, the subject has not been previously treated with a PD-1 inhibitor monotherapy.

As used herein, the terms “cancer” and “cancerous” refer to or describe the physiological condition in which a population of cells is characterized by unregulated cell growth.

In carrying out the methods disclosed herein, the cancer may be a carcinoma, sarcoma, melanoma, leukemia, lymphoma, or combinations thereof (mixed-type cancer). The term “carcinoma” refers to a cancer originating from epithelial cells of the skin or the lining of the internal organs. The term “sarcoma” refers to a tumor derived from mesenchymal cells, usually those constituting various connective tissue cell types, including fibroblasts, osteoblasts, endothelial cell precursors, and chondrocytes. The term “melanoma” refers to a tumor arising from melanocytes, the pigmented cells of the skin and iris. In some embodiments, the cancer is not a melanoma. The term “leukemia” refers to a malignancy of any of a variety of hematopoietic stem cell types, including the lineages leading to lymphocytes and granulocytes, in which the tumor cells are nonpigmented and dispersed throughout the circulation. The term “lymphoma” refers to a solid tumor of the lymphoid cells and includes a variety of disease states, e.g., non-Hodgkins lymphoma (NHL); diffuse large B-cell lymphoma (DLBCL); follicular lymphoma (FL); Hodgkin's disease; Burkitt's lymphoma; cutaneous T-cell lymphoma (CTCL); primary central nervous system lymphoma, and lymphomatous metastases.

Suitable T cell lymphomas include cutaneous T-cell lymphoma (CTCL) and peripheral T-cell lymphoma (PTCL). As described herein, CTCL is a type of non-Hodgkin's lymphoma of primary cutaneous disease with various other manifestations in additional sites like lymph nodes and peripheral blood. In CTCL, some of the T cells (a type of lymphocyte involved in the immune system) become cancerous, causing skin lesions and reducing the patient's QOL (Quality of Life) due to pain and pruritus. CTCL is generally a low-grade lymphoma with initial patch and plaque skin lesions, but it progresses slowly and advances to the tumor stage over several years to over a dozen years. CTCL is still a disease with extremely high unmet medical needs because it has a high malignancy when it reaches the tumor stage and has a poor prognosis.

In some embodiments, the cancer is a CTCL selected from the group consisting of, e.g., mycosis fungoides (MF), Sézary syndrome (SS), and primary cutaneous CD30⁺ lymphoproliferative disorder (LPD), or variants thereof. See, e.g., Willemze et al., “The 2018 Update of the WHO-EORTC Classification for Primary Cutaneous Lymphomas,” Blood 133(16): 1703-1714 (2019), which is hereby incorporated by reference in its entirety.

MF encompasses 50-60% of all CTCLs. MF is skin limited, and leukemic involvement occurs only in few cases progressing to advanced disease (see, e.g., Walia & Yeung, “An Update on Molecular Biology of Cutaneous T Cell Lymphoma,” Front. Oncol. 9: 1558 (2019), which is hereby incorporated by reference in its entirety). MF T cells are T resident memory (Trm) cells exhibiting CCR4⁺/CLA⁺/L-selectin⁻/CCR7⁻. Trm cells are skin tropic and stay within the epithelial barriers. MF passes through various clinical stages and early lesions (eMF). Affected individuals may first develop a red rash or dry, red, scaly patches of skin that most often affect the buttocks and trunk (premyotic phase). These patches may remain unchanged, spontaneously go away, or slowly grow larger. The skin lesions associated with the initial phase of MF are termed “nonspecific” because they cannot be differentiated from skin lesions associated with other, more common, skin disorders such as psoriasis. This initial phase of MF may persist for months, years, or decades. In the second phase of MF, slightly-elevated, reddish-brown, scaly bumps (plaques) develop on the skin (mycotic stage). These plaques may develop from existing patches or spontaneously in unaffected areas. Eventually, these plaques may expand and grow together (coalesce), forming larger plaques. Any area of the body may be affected. The skin lesions associated with the first two phases of MF may not be associated with other symptoms (asymptomatic) or may occur along with itchiness (pruritis) and pain. In rare cases, affected individuals may experience difficulty sleeping due to severe itchiness. The third phase of MF is characterized by the development of mushroom-shaped tumors. In some cases, the tumors may become ulcerated and infected. Some individuals may not progress beyond the plaque phase of MF and do not develop tumors. Other individuals may develop tumors without first developing the patches or plaques associated with the early stages of MF. In some individuals with MF, malignant lymphocytes may spread beyond the skin to affect the lymph nodes and major organs of the body, including the liver, spleen, and gastrointestinal system.

SS is the leukemic variant of MF, presenting with erythrodermic lesions along with lymph node and peripheral blood involvement at presentation (see, e.g., Walia & Yeung, “An Update on Molecular Biology of Cutaneous T Cell Lymphoma,” Front. Oncol. 9: 1558 (2019), which is hereby incorporated by reference in its entirety). SS malignant T cells are central memory cells (Tcm) (CCR4⁺/L-selectin⁺/CCR7⁺). Tcm cells have the ability to shuffle between skin, lymph nodes, and blood. SS is characterized by a widespread red rash that may cover most of the body (generalized erythroderma), the presence of specific malignant lymphocytes (Sezary cells) in the blood, and abnormally enlarged lymph nodes (lymphadenopathy). Individuals with SS may experience intense itchiness (pruritis) and thickening, scaling, and peeling (exfoliation) of the skin. Additional symptoms associated with SS include outward turning of the eyelids (ectropion); abnormally thick, rough skin on the palms of the hands and the soles of the feet (palmoplantar keratoderma); malformation of the nails (onychodystrophy); and abnormal enlargement of the liver and/or spleen (hepatosplenomegaly). General symptoms associated with SS include fevers, weight loss, bald patches on the scalp (alopecia), and a general feeling of ill health (malaise).

Folliculotropic MF (FMF), pagetoid reticulosis (PR), and granulomatous slack skin (GSS) are recognized as distinct variants of MF, because of their distinctive clinicopathologic features, clinical behavior, and/or prognosis (Willemze et al., “The 2018 Update of the WHO-EORTC Classification for Primary Cutaneous Lymphomas,” Blood 133(16): 1703-1714 (2019), which is hereby incorporated by reference in its entirety). Whereas PR and GSS are extremely rare, FMF accounts for ˜10% of all cases of MF.

FMF differs from the classic form of MF by the presence of folliculotropic infiltrates, often with sparing of the epidermis, the preferential localization of skin lesions in the head and neck region, and the presence of (grouped) follicular papules, acneiform lesions, and associated alopecia.

PR, also known as Woringer-Kolopp disease, is a rare skin condition characterized by a solitary lesion that usually affects the arms or legs and may grow slowly.

GSS is characterized by areas (folds) of lax, reddened skin. The underarms, groin, and stomach are most often affected. GSS is usually a benign, slow-growing (indolent) form of CTCL.

Primary cutaneous CD30⁺ LPDs are the second most common group of CTCL, accounting for ˜25% of all CTCLs. This group includes primary cutaneous anaplastic large lymphoma (C-ALCL) and lymphomatoid papulosis (LyP), which form a spectrum of diseases. Because of the overlapping histologic and phenotypic features, clinical presentation and clinical course are used as decisive criteria to differentiate between LyP and C-ALCL.

In some embodiments, the CTCL is C-ALCL. C-ALCL presents as solitary, grouped, or, uncommonly, multifocal nodules and tumors. Cutaneous relapses are common, but extracutaneous dissemination occurs in only 10% to 15% of patients. Affected individuals develop tumors on the skin, which may become ulcerated or infected. In some cases, the lesions or tumors go away without treatment (spontaneous regression). However, lesions often return (relapse). In rare cases, other organ systems of the body may become involved.

LyP is characterized by a chronic course of recurrent, self-healing papulonecrotic or nodular skin lesions that most often affect the trunk, face, arms, and legs. These lesions often become crusted or ulcerated, sometimes leaving a scar.

Additional suitable CTCL includes, without limitation, adult T cell leukemia/lymphoma; extranodal NK/T cell lymphoma, nasal type; chronic active EBV infection; primary cutaneous peripheral T-cell lymphoma, rare subtypes; and primary cutaneous peripheral T-cell lymphoma, NOS (Willemze et al., “The 2018 Update of the WHO-EORTC Classification for Primary Cutaneous Lymphomas,” Blood 133(16): 1703-1714 (2019), which is hereby incorporated by reference in its entirety).

As described herein, PTCL is a type of T-cell non-Hodgkin's lymphoma that is classified as an intermediate-grade lymphoma. PTCL is often detected in advanced stages and has symptoms such as swelling and lumps in the lymph nodes, fever, heavy night sweats, and weight loss. Among PTCLs, Anaplastic Lymphoma Kinase (ALK)-positive anaplastic large cell lymphoma, which occurs in the 20s and 30s, has a favorable prognosis and is curable. However, other types of PTCL often occur around the age of 60, and may have a poor prognosis or be difficult to treat. Therefore, PTCL is still a disease with extremely high-unmet medical needs.

Suitable PTCLs include, e.g., peripheral T-cell lymphoma not otherwise specified (PTCL-NOS), angioimmunoblastic T-cell lymphoma (AITL), anaplastic large cell lymphoma, adult T-cell leukemia/lymphoma (ATLL), and enteropathy-associated T-cell lymphoma (EATL).

PTCL-NOS, also referred to as PCTL-U or PTCL-unspecified, are aggressive lymphomas, mainly of nodal type, but extranodal involvement is common. The majority of nodal cases are CD4⁺ and CD8⁻, and CD30 can be expressed in large cell variants. Most patients with PTCL-NOS present with nodal involvement; however, a number of extranodal sites may also be involved (e.g., liver, bone marrow, gastrointestinal tract, skin). Studies generally report a 5-year overall survival of approximately 30%-35% using standard chemotherapy.

AITL is an unusual subtype of mature peripheral T-cell lymphoma originating from the follicular T helper cells and is often associated with autoimmune disorders (see, e.g., Kanderi et al., “Angioimmunoblastic T-cell Lymphoma: An Unusual Case in an Octogenarian,” Cureus 12(2): e6956 (2020), which is hereby incorporated by reference in its entirety). AITL is an aggressive lymphoma, presenting with constitutional symptoms, generalized lymphadenopathy and hepatosplenomegaly. Immunohistochemistry and biopsy are diagnostic methods. The treatment modalities range from steroids, immunomodulators, and cytotoxic chemotherapy. AITL constitutes approximately 1% to 2% of non-Hodgkin's lymphoma and about 15% to 20% of peripheral T-cell lymphoma.

ALCL represents a group of malignant T cell lymphoproliferation that share morphological and immunophenotypical features, namely strong CD30 expression and variable loss of T cell markers, but differ in clinical presentation and prognosis (see, e.g., Montes-Mojarro et al., “The Pathological Spectrum of Systemic Anaplastic Large Cell Lymphoma (ALCL),” Cancers (Basel) 10(4): 107 (2018), which is hereby incorporated by reference in its entirety). The recognition of anaplastic lymphomakinase (ALK) fusion proteins as a result of chromosomal translocations or inversions was the starting point for the distinction of different subgroups of ALCL. According to their distinct clinical settings and molecular findings, the 2016 revised World Health Organization (WHO) classification recognizes four different entities: systemic ALK-positive ALCL (ALK⁺ ALCL), systemic ALK-negative ALCL (ALK⁻ ALCL), primary cutaneous ALCL (pC-ALCL), and breast implant-associated ALCL (BI-ALCL), the latter included as a provisional entity. ALK is rearranged in approximately 80% of systemic ALCL cases with one of its partner genes, most commonly NPM1, and is associated with favorable prognosis, whereas systemic ALK⁻ ALCL shows heterogeneous clinical, phenotypical, and genetic features, underlining the different oncogenesis between these two entities.

ALK⁺ ALCL is a type of PTCL consisting of large lymphoid cells with abundant cytoplasm and pleomorphic, often horseshoe-shaped nuclei, characterized by strong CD30 immunostaining and ALK chromosomal translocation (see, e.g., Montes-Mojarro et al., “The Pathological Spectrum of Systemic Anaplastic Large Cell Lymphoma (ALCL),” Cancers (Basel) 10(4): 107 (2018), which is hereby incorporated by reference in its entirety).

Systemic ALK⁺ ALCL (sALK⁺ ALCL) predominantly occurs in children and young adults with a slight male predominance. sALK⁺ ALCL shows an aggressive behavior with rapidly progressive adenopathy and systemic symptoms such as fevers, night sweats, and weight loss. At the time of diagnosis, most patients are in an advanced stage of disease (III-IV stage) with systemic symptoms (75%) and lymph node enlargement (90%), including mediastinal involvement (36%). Extranodal involvement is present in 40-68% of cases, including skin (26%), bone (14%), and soft tissues (15%), lung (12%), and liver (8%).

Systemic ALK⁻ ALCL (s ALK⁻ ALCL) has similar morphology and phenotype to ALK⁺ ALCL, but by definition, lacks ALK rearrangement and ALK expression. ALK⁻ ALCL usually affects adults with a slight male predominance; the mean age of diagnosis is between 55 and 60 years. Half of the cases involve lymph nodes, and only 20% of the cases show an extranodal presentation.

ATLL is an aggressive T cell neoplasm arising from post-thymic regulatory T cells and caused by the oncoretrovirus human T cell leukemia virus type 1 (HTLV-1). ATLL occurs in approximately 3%-5% of HTLV-1 carriers during their lifetime and follows a heterogeneous clinical course (see, e.g., Kato & Akashi, “Recent Advances in Therapeutic Approaches for Adult T-Cell Leukemia/Lymphoma,” Viruses 7(12): 6604-6612 (2015), which is hereby incorporated by reference in its entirety). ATLL is characterized by a high tendency for leukemic changes and involves various organs, including the GI tract, liver, spleen, and skin.

EATL is a lethal type of peripheral T cell lymphoma that is the most common oncologic complication of celiac disease, with a prevalence of ˜1% in those patients (see, e.g., Moffitt et al., “Enteropathy-Associated T cell Lymphoma Subtypes are Characterized by Loss of Function of SETD2,” J. Exp. Med. 214(5): 1371-1386 (2017), which is hereby incorporated by reference in its entirety). There are two recognized subtypes of EATL. Type I EATL has a more variable histology, is more prevalent in Northern Europe, and is strongly associated with celiac disease. Type II EATL has more uniform histology, occasional association with celiac disease, and is more prevalent in Asia. Cases are currently classified based on their morphology and immunophenotype, with both types sharing common T cell markers but type II cases expressing CD56 more frequently.

Additional exemplary cancers include, e.g., acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), mixed-cell carcinoma, mucinous (colloid) carcinoma, mucinous cystadenocarcinoma, papillary adenocarcinoma, pleomorphic giant-cell carcinoma, serous cystadenocarcinoma, and small-cell (oat-cell) carcinoma.

Cancers may be named according to the organ in which they originate. Thus, in some embodiments, the cancer is selected from the group consisting of breast cancer, uterine corpus cancer, cervical cancer, ovarian cancer, prostate cancer, lung cancer, stomach cancer, non-small cell lung cancer, spleen cancer, head and neck squamous cell carcinoma, esophageal cancer, bladder cancer, melanoma, colorectal cancer, kidney cancer, non-Hodgkin lymphoma, urothelial cancer, sarcoma, blood cell carcinoma, bile duct carcinoma, gallbladder carcinoma, thyroid carcinoma, prostate cancer, testicular carcinoma, thymic carcinoma, and hepatocarcinoma.

As described herein, the inhibitory checkpoint receptor PD-1 is expressed on activated T-cells, B-cells, and myeloid cells. The binding of PD-1 to PD-L1 expressed on the surface of cancer/tumor cells results in suppression of proliferation and the immune response of the T effector cells. Thus, activation of the PD-1/PD-L1 signal pathway serves as a major mechanism of immune evasion by cancer/tumor cells. In some embodiments, the cancer is a PD-L1 positive (PD-L1⁺) cancer. The term “PD-L1 positive cancer” refers to a cancer comprising cells that express PD-L1 (also known as CD274, PDCD1L1, or B7-H1).

Cancer may be resistant to cytotoxic fusion protein monotherapy or PD-1 pathway inhibitor monotherapy. As used herein, the term “resistant” refers to a condition that results when a cancer becomes tolerant to the administration of a particular therapeutic agent (e.g., a cytotoxic fusion protein or a PD-1 pathway inhibitor). See, e.g., Houseman et al., “Drug Resistance in Cancer: An Overview,” Cancers (Basel) 6(3): 1769-1792 (2014), which is hereby incorporated by reference in its entirety).

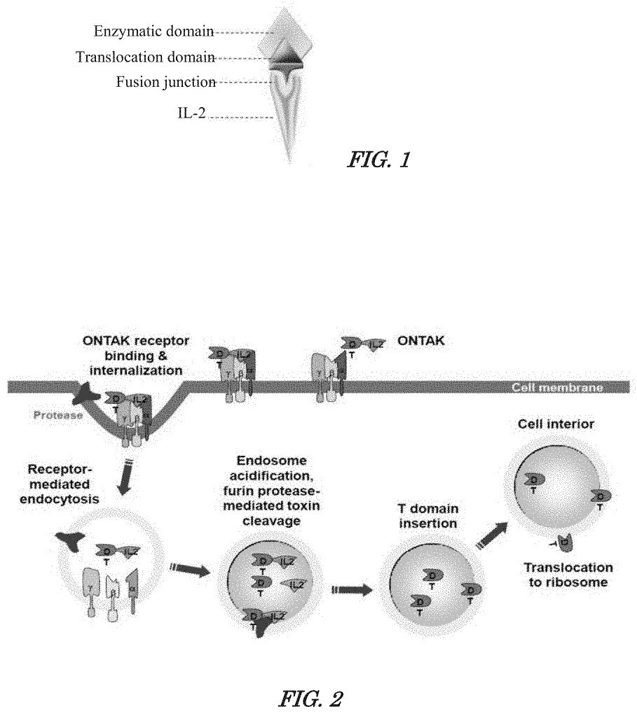

FIG. 1 provides a schematic illustration of a cytotoxic fusion protein comprising an N-terminus coupled to a C-terminus, where the N-terminus comprises amino acid sequences of diphtheria toxin fragments A and B, and the C-terminus comprises amino acid sequences corresponding to human interleukin-2 (IL-2). The C-terminus of the cytotoxic fusion protein mediates binding to the IL-2 receptor (IL-2R) on T-cell lymphoma cells. As described herein, the N-terminus of the cytotoxic fusion protein directs the cytocidal action of diphtheria toxin to cells which express the IL-2 receptor (e.g., T cells).

In some embodiments, the monomeric cytotoxic fusion protein for use in the methods described herein comprises the amino acid sequence of SEQ ID NO:1, as shown in Table 1 below.

```
TABLE 1
Denileukin diftitox (CAS Registry Number: 173146-27-5)
CA Index Name: 1-388-Toxin, (Corynebacterium diphtheriae
  strain C7), N-L-methionyl-387-L-histidine-388-L-alanine-,
(388 → 2′)-protein with 2-133-interleukin 2 (human clone
    pTIL2-21a)
```

| | |
|---|---|
| 1: | MGADDVVDSS KSFVMENFSS YHGTKPGYVD SIQKGIQKPK SGTQGNYDDD |
| 51: | WKGFYSTDNK YDAAGYSVDN ENPLSGKAGG VVKVTYPGLT KVLALKVDNA |
| 101: | ETIKKELGLS LTEPLMEQVG TEEFIKRFGD GASRVVLSLP FAEGSSSVEY |
| 151: | INNWEQAKAL SVELEINFET RGKRGQDAMY EYMAQACAGN RVRRSVGSSL |
| 201: | SCINLDWDVI RDKTKTKIES LKEHGPIKNK MSESPNKTVS EEKAKQYLEE |
| 251: | FHQTALEHPE LSELKTVTGT NPVFAGANYA AWAVNVAQVI DSETADNLEK |
| 301: | TTAALSILPG IGSVMGIADG AVHHNTEEIV AQSIALSSLM VAQAIPLVGE |
| 351: | LVDIGFAAYN FVESIINLFQ VVHNSYNRPA YSPGHKTHAP TSSSTKKTQL |
| 401: | QLEHLLLDLQ MILNGINNYK NPKLTRMLTF KFYMPKKATE LKHLQCLEEE |
| 451: | LKPLEEVLNL AQSKNFHLRP RDLISNINVI VLELKGSETT FMCEYADETA |
| 501: | TIVEFLNRWI TFCQSIISTL T (SEQ ID NO: 1) |

Sequence Notes:

| | | |
|---|---|---|
| Type | Location | Description |
| bridge | CYS-202-CYS-187 | disulfide bridge |
| bridge | CYS-493-CYS-446 | disulfide bridge |

In some embodiments, the monomeric cytotoxic fusion protein does not comprise a modification (e.g., a substitutions, deletion, and/or insertion) at any one of amino acid residues 1-521 of SEQ ID NO:1. In some embodiments, the cytotoxic fusion protein does not comprise a modification (e.g., a substitutions, deletion, and/or insertion) at amino acid residue 6 of SEQ ID NO:1. For example, in some embodiments, the cytotoxic fusion protein does not comprise a V6A substitution in SEQ ID NO:1.

In some embodiments, the monomeric cytotoxic fusion protein is denileukin diftitox (DD) (CAS Reg. No. 173146-27-5) (see, e.g., U.S. Pat. Nos. 5,763,250; 5,703,039; and 6,074,636, each hereby incorporated by reference in its entirety). DD is a recombinant DNA-derived cytotoxic fusion protein composed of the amino acid sequences for diphtheria toxin fragments A and B (Met₁-Thr₃₈₇)-His and the sequence for human interleukin-2 (IL-2; Alai-Thr₁₃₃). Expression of DD in E. coli and subsequent purification from inclusion bodies may result in the presence of various species, which include a 58 kD active monomer fusion protein and various impurities (e.g., fusion protein aggregates, which can include inactive and misfolded species). Ontak® is a pharmaceutical composition comprising DD in a sterile solution of citric acid (20 mM), EDTA (0.05 mM), and polysorbate 20 (<1%) in water (pH range of 6.9 to 7.2). The total cytotoxic fusion protein content of Ontak® has been reported to contain approximately 40% protein aggregates.

In some embodiments, the monomeric cytotoxic fusion protein is provided in a pharmaceutical formulation commercially available as Remitoro® in Japan. As described herein, Remitoro® (E7777) comprises a fusion protein consisting of interleukin-2 (IL-2) and a partial sequence of diphtheria toxin, and specifically binds to the IL-2 receptor on the surface of tumoral lymphocytes. The antitumor effect of Remitoro® depends on the intracellular delivery of diphtheria toxin fragment which inhibits protein synthesis and induces cell death (see “Anticancer Agent Remitoro® Intravenous Drip Infusion 300 μg′ (Denileukin Diftitox (Genetic Recombinant) Approved in Japan for Peripheral T-Cell Lymphoma and Cutaneous T-Cell Lymphoma,” available at https://www.esai.com/news/2021/news202119.html, which is hereby incorporated by reference in its entirety).

E7777 was evaluated in a multicenter, open-label, single-arm phase II clinical study to determine the efficacy and safety in patients with relapsed or refractory Peripheral T cell Lymphoma (PTCL) or Cutaneous T-cell Lymphoma (CTCL). Patients who participated in the study received a final histopathological definitive diagnosis by the Central Committee for Pathological Diagnosis, which is independent of the clinical study site. The histopathological subtypes of participants consisted of 17 patients with PTCL, 19 patients with CTCL, and 1 patient with another malignant lymphoma. The efficacy of the agent was evaluated in 36 patients with PTCL or CTCL, and the safety was evaluated in 37 patients. The agent was administered by intravenous drip infusion over 60 minutes at a dose of 9 μg/kg/day for five consecutive days from day 1 to day 5 to complete a cycle, with one cycle every three weeks and a maximum of up to 8 cycles conducted. In this study, the primary endpoint was the objective response rate, and the efficacy of the agent was evaluated on the basis that the lower limit of the confidence interval (CI) was above a predetermined threshold. The study achieved the primary endpoint target and exceeded a pre-specified tumor response threshold with statistical significance: the objective response rate (ORR) of PTCL and CTCL patients in total (n=36) was 36.1% (95% confidence interval (CI): 20.8-53.8). The ORRs of each subtype were 41.2% (95% CI: 18.4-67.1) for PTCL (n=17) and 31.6% (95% CI: 12.6-56.6) for CTCL (n=19). The five most frequent treatment-emergent adverse events observed in the study were increased aspartate aminotransferase (AST) (89.2%), increased alanine aminotransferase (ALT) (86.5%), hypoalbuminaemia (70.3%), lymphopenia (70.3%), and pyrexia (51.4%).

As described herein, the present composition comprising the monomeric cytotoxic fusion protein may be a pharmaceutical composition. The pharmaceutical composition may contain a greater proportion of the active monomeric species of DD than present in Ontak®. Thus, in some embodiments, at least 95.0%, 95.5%, 96%, 96.5%, 97%, 97.5%, 98%, 98.5%, 99%, 99.5%, or more of the total cytotoxic fusion protein content of the pharmaceutical composition described herein is the active monomeric species of DD. In some embodiments, the pharmaceutical composition, according to the present disclosure, is a lyophilized pharmaceutical composition comprising a greater proportion of the active monomeric species of DD than present in Ontak®. In some embodiments, the pharmaceutical composition according to the present disclosure is a lyophilized pharmaceutical composition, where at least 95.0%, 95.5%, 96%, 96.5%, 97%, 975%, 98%, 98.5%, 99%, 99.5%, or more of the total cytotoxic fusion protein content is the active monomeric species of DD. In some embodiments, the pharmaceutical composition may be formulated for intravenous administration, e.g., intravenous pump infusion or intravenous drip infusion.

The composition comprising the monomeric cytotoxic fusion proteins according to the present disclosure may comprise isolated monomeric cytotoxic fusion proteins or polypeptides. In some embodiments, the isolated monomeric cytotoxic fusion proteins of the present disclosure are prepared for use in the methods disclosed herein using standard methods of synthesis known in the art, including solid-phase peptide synthesis (Fmoc or Boc strategies) or solution phase peptide synthesis. Alternatively, monomeric cytotoxic fusion proteins of the present disclosure may be prepared using recombinant expression systems.

Traditional strategies for recombinant protein expression involve transfecting cells with a DNA vector that contains the template and then culturing the cells so that they transcribe and translate the desired protein. Cells may then be lysed to extract the expressed protein for subsequent purification. Suitable expression systems are well known in the art and include, without limitation, mammalian cell expression systems, insect cell expression systems, yeast cell expression systems, bacterial cell expression systems (e.g., an E. coli expression system), algal cell expression systems, and cell-free expression systems (see, e.g., U.S. Pat. No. 5,703,039 to Williams et al., which is hereby incorporated by reference in its entirety).

Purified monomeric cytotoxic fusion proteins may be obtained by several methods readily known in the art, including ion-exchange chromatography, hydrophobic interaction chromatography, affinity chromatography, gel filtration, and reverse-phase chromatography. The protein is preferably produced in purified form (preferably at least about 80% or 85% pure, more preferably at least about 90% or 95% pure) by conventional techniques. Depending on whether the recombinant host cell is made to secrete the protein into a growth medium (see U.S. Pat. No. 6,596,509 to Bauer et al., which is hereby incorporated by reference in its entirety), the cytotoxic fusion protein can be isolated and purified by centrifugation (to separate cellular components from supernatant containing the secreted protein) followed by sequential ammonium sulfate precipitation of the supernatant. The fraction containing the cytotoxic fusion protein is subjected to gel filtration in an appropriately sized dextran or polyacrylamide column to separate the protein of interest from other proteins. If necessary, the protein fraction may be further purified by HPLC.

As used herein, PD-1 inhibitors include inhibitors of the PD-1 pathway. The programmed cell death-1 receptor (PD-1) is a receptor on T-cells that inhibits signaling downstream of the T-cell Receptor (TCR) as well as other T-cell co-receptors. Therefore, signal transduction initiated via its ligands, PD-L1 or PD-L2 (programmed cell death 1 ligand 1 and 2), usually provides a suppressive or inhibitory signal to the T-cell (e.g., a regulatory T cell) that results in decreased T-cell proliferation or other inhibition of T-cell functions. Since the PD-1/PD-L1/PD-L2 axis is a critical immune checkpoint that tips immune responses towards tolerance, the PD-1/PD-L1 receptor-ligand pair has been heavily targeted in cancer immunotherapy with monoclonal antibody therapies aimed to block their interaction.

Members of the PD-1 pathway include proteins that are associated with PD-1 signaling. Such proteins include those that induce PD-1 signaling upstream of PD-1 as, e.g., ligands of PD-1, PD-L1, and PD-L2 and the signal transduction receptor PD-1. Such proteins also include signal transduction proteins downstream of PD-1 receptor. Exemplary members of the PD-1 pathway in the context of the present disclosure include PD-1, PD-L1, and PD-L2.

A PD-1 pathway inhibitor includes compound(s) capable of impairing PD-1 pathway signaling. A PD-1 pathway inhibitor may be any inhibitor directed against any member of the PD-1 pathway capable of antagonizing PD-1 pathway signaling. In this context, the PD-1 pathway inhibitor may be an antagonistic antibody as defined herein, targeting any member of the PD-1 pathway, e.g., against PD-1 receptor, PD-L1, or PD-L2. This antagonistic antibody may also be encoded by a nucleic acid. Such encoded antibodies are also called “intrabodies,” as defined herein. Also, the PD-1 pathway inhibitor may be a fragment of the PD-1 receptor or the PD1-receptor blocking the activity of PD1 ligands. B7-1 or fragments thereof may act as PD1-inhibiting ligands as well. Furthermore, the PD-1-inhibitor may be siRNA (small interfering RNA) or antisense RNA directed against a member of the PD-1 pathway, preferably PD-1, PD-L1, or PD-L2. Additionally, a PD-1-inhibitor may be a protein comprising (or a nucleic acid coding for) an amino acid sequence capable of binding to PD-1 but preventing PD-1 signaling, e.g., by inhibiting PD-1 and B7-H1 or B7-DL interaction. Additionally, a PD-1 pathway inhibitor may be a small molecule inhibitor capable of inhibiting PD-1 pathway signaling, e.g., a PD-1 binding peptide or a small organic molecule.

Suitable PD-1 pathway inhibitors include, without limitation, anti-PD-1 antibody, anti-PD-L1 antibody, anti-PD-L2 antibody, anti-PD-1 RNAi, anti-PD-L1 RNAi, anti-PD-L2 RNAi, anti-PD-1 antisense RNA, anti-PD-L1 antisense RNA, anti-PD-L2 antisense RNA, dominant negative PD-1 protein, dominant negative PD-L1 protein, dominant negative PD-L2 protein, and small molecule inhibitors (see, e.g., U.S. Pat. Pub. No. 2018/0362650, hereby incorporated by reference in its entirety).

In some embodiments, the PD-1-inhibitor is an antibody. An antibody may be selected from any antibody, e.g., any recombinantly produced or naturally occurring antibodies, known in the art. Exemplary antibodies include those suitable for therapeutic, diagnostic, or scientific purposes directed against PD-1, PD-L1, or PD-L2. The term “antibody” is used herein in its broadest sense encompasses monoclonal and polyclonal antibodies (including antagonist and blocking or neutralizing antibodies) and antibody species with polyepitopic specificity. As used herein, the term “antibody” comprises any antibody known in the art (e.g., IgM, IgD, IgG, IgA, and IgE antibodies), such as naturally occurring antibodies, antibodies generated by immunization in a host organism, antibodies that were isolated and identified from naturally occurring antibodies or antibodies generated by immunization in a host organism and recombinantly produced by biomolecular methods known in the art, as well as chimeric antibodies, human antibodies, humanized antibodies, bispecific antibodies, intrabodies, i.e., antibodies expressed in cells and optionally localized in specific cell compartments, and fragments and variants of the aforementioned antibodies. In general, an antibody consists of a light chain and a heavy chain, both having variable and constant domains. The light chain consists of an N-terminal variable domain, VL, and a C-terminal constant domain, CL. In contrast, the heavy chain of the IgG antibody, for example, is comprised of an N-terminal variable domain, VH, and three constant domains, CH1, CH2, and CH3. In some embodiments, the antibody is a single-chain antibody.

In some embodiments, the antibodies comprise full-length antibodies, i.e., antibodies composed of the full heavy and full light chains, as described above. However, derivatives of antibodies such as antibody fragments, variants, or adducts may also be used as a PD-1-inhibitor. Antibody fragments may be selected from Fab, Fab′, F(ab′)2, Fc, Facb, pFc′, Fd and Fv fragments of the aforementioned (full-length) antibodies. In general, antibody fragments are known in art. For example, a Fab (“fragment, antigen-binding”) fragment is composed of one constant and one variable domain of each of the heavy and the light chain. The two variable domains bind the epitope on specific antigens. The two chains are connected via a disulfide linkage. An scFv (“single-chain variable fragment”) fragment, for example, typically consists of the variable domains of the light and heavy chains. The domains are linked by an artificial linkage, in general, a polypeptide linkage such as a peptide composed of 15-25 glycine, proline and/or serine residues.

In some embodiments, the antibody is a polyclonal antibody. The term “polyclonal antibody” refers to mixtures of antibodies directed to specific antigens or immunogens or epitopes of a protein which were generated by immunization of a host organism, such as a mammal, e.g., including goat, cattle, swine, dog, cat, donkey, monkey, ape, a rodent such as a mouse, hamster, and rabbit. Polyclonal antibodies are generally not identical, and thus usually recognize different epitopes or regions from the same antigen. Thus, in such a case, typically, a mixture (a composition) of different antibodies will be used, each antibody being directed to specific antigens or immunogens or epitopes of a protein, particularly directed to PD-1, PD-L1, or PD-L2.

In some embodiments, the antibody is a monoclonal antibody. As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed to a single antigenic site. Furthermore, in contrast to conventional (polyclonal) antibody preparations, which typically include different antibodies directed to different determinants (epitopes), each monoclonal antibody is directed to a single determinant on the antigen. For example, monoclonal antibodies as defined above may be made by the hybridoma method first described by Kohler and Milstein, Nature, 256:495 (1975), or may be made by recombinant DNA methods, e.g., as described in U.S. Pat. No. 4,816,567. “Monoclonal antibodies” may also be isolated from phage libraries generated using the techniques described in McCafferty et al., Nature, 348:552-554 (1990), for example. According to Kohler and Milstein, an immunogen (antigen) of interest is injected into a host such as a mouse, and B-cell lymphocytes produced in response to the immunogen are harvested after a period of time. The B-cells are combined with myeloma cells obtained from mouse and introduced into a medium that permits the B-cells to fuse with the myeloma cells, producing hybridomas. These fused cells (hybridomas) are then placed into separate wells of microtiter plates and grown to produce monoclonal antibodies. The monoclonal antibodies are tested to determine which of them are suitable for detecting the antigen of interest. After being selected, the monoclonal antibodies can be grown in cell cultures or by injecting the hybridomas into mice. Suitable antibodies include, e.g., monoclonal antibodies directed against PD-1, PD-L1, and PD-L2.

In some embodiments, the antibody is a chimeric antibody. Chimeric antibodies, which may be used as PD-1 pathway inhibitors according to the methods described herein, are antibodies in which the constant domains of an antibody described above are replaced by sequences of antibodies from other organisms, e.g., human sequences.

In some embodiments, the antibody is a humanized antibody. Humanized (non human) antibodies, which may be used as PD-1 pathway inhibitors according to the methods described herein, are antibodies in which the constant and variable domains (except for the hypervariable domains) of an antibody are replaced by human sequences.

In some embodiments, the antibody is a human antibody. Human antibodies can be isolated from human tissues or from immunized non-human host organisms, which are transgene for the human IgG gene locus. Additionally, human antibodies can be provided by the use of a phage display.

In some embodiments, the antibody is a bispecific antibody. Bispecific antibodies in the context of the methods described herein are antibodies that act as an adaptor between an effector and a respective target by two different Fa/b-domains, e.g., for the purposes of recruiting effector molecules such as toxins, drugs, cytokines, etc., targeting effector cells such as CTL, NK cells, macrophages, granulocytes, etc. (see, e.g., Kontermann R. E., Acta Pharmacol. Sin, 2005, 26(1): 1-9). Bispecific antibodies as described herein are, in general, configured to recognize by two different Fa/b-domains, e.g., two different antigens, immunogens, epitopes, drugs, cells (or receptors on cells), or other molecules (or structures) as described above. Bispecificity means herewith that the antigen-binding regions of the antibodies are specific for two different epitopes. Thus, different antigens, immunogens or epitopes, etc., can be brought close together, which, optionally, allows direct interaction of the two components. For example, different cells such as effector cells and target cells can be connected via a bispecific antibody. Encompassed, but not limited, by the present disclosure are antibodies or fragments thereof which bind, on the one hand, a soluble antigen and, on the other hand, an antigen or receptor e.g., PD-1 or its ligands PD-L1 and PD-L2 on the surface of a target cell, e.g., a tumor cell.

In some embodiments, the antibody is an intrabody. Intrabodies are intracellular expressed antibodies, and therefore these antibodies may be encoded by nucleic acids to be used for the expression of the encoded antibodies. Therefore nucleic acids coding for an antibody, preferably as defined above, particularly an antibody directed against a member of the PD-1 pathway, e.g., PD-1, PD-L1, or PD-L2 may be used as PD-1-inhibitor according to the methods described herein.

In carrying out the methods described herein, the PD-1 pathway inhibitor may be an antibody. For example, the PD-1 pathway inhibitor may be an anti-PD-1 antibody. Suitable anti-PD-1 antibodies include, without limitation, nivolumab (OPDIVO®), pembrolizumab (KEYTRUDA®), cemiplimab (LIBTAYO®), pidilizumab (CT-011), REGN2810 (SAR-439684), spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514 (MEDI0680), and PF-06801591 (see, e.g., Liao et al., “A Review of Efficacy and Safety of Checkpoint Inhibitor for the Treatment of Acute Myeloid Leukemia,” Front. Pharmacol. 10: 609 (2019), which is hereby incorporated by reference in its entirety). Nivolumab (OPDIVO®) is a fully human IgG4 monoclonal antibody; pembrolizumab (KEYTRUDA®) is a humanized monoclonal IgG4 antibody; pidilizumab (CT-011) is a humanized, IgG1 monoclonal antibody; REGN2810 (SAR-439684) is a human monoclonal antibody; spartalizumab (PDR 001) is a humanized monoclonal antibody; camrelizumab (SHR-1210) is a monoclonal antibody, and MEDI0680 is a humanized IgG4 monoclonal antibody directed against PD-1.

The PD-1 pathway inhibitor may be an anti-PD-L1 antibody. Suitable anti-PD-L1 antibodies include, without limitation, atezolizumab (TECENTRIQ®), avelumab (BAVENCIO®), durvalumab (IMFINZI®), KN035, CK-301, AUNP12, CA-170, BMS-986189, MPDL3280A, and MEDI4736 (see, e.g., Powles et al., “MPDL3280A (anti-PD-L1) Treatment Leads to Clinical Activity in Metastatic Bladder Cancer,” Nature 515(7528): 558-62 (2014) and Massard et al., “Safety and Efficacy of Durvalumab (MEDI4736), an Anti-Programmed Cell Death Ligand-1 Immune Checkpoint Inhibitor, in Patients With Advanced Urothelial Bladder Cancer,” J. Clin. Oncol. 34(26):3119-3125 (2016), which are hereby incorporated by reference in their entirety).

In practicing this and other aspects of the present application that involve administering therapeutic agents to a subject (e.g., a cytotoxic fusion protein and a PD-1 pathway inhibitor), the therapeutic agents may be administered before, after, or simultaneously with the administration of any, some, or all of the other therapeutic agents described herein. Thus, in some embodiments, said administering the recombinant cytotoxic fusion protein is carried out before, after, or simultaneously with the administration of the PD-1 pathway inhibitor. In some embodiments, the therapeutic agents may be administered by the same route of administration, or the therapeutic agents may be administered by different routes of administration.

For example, (i) cytotoxic fusion protein and (ii) PD-1 pathway inhibitor(s) can be administered about one week apart, about 6 days apart, about 5 days apart, about 4 days apart, about 3 days apart, about 2 days apart, about 24 hours apart, about 23 hours apart, about 22 hours apart, about 21 hours apart, about 20 hours apart, about 19 hours apart, about 18 hours apart, about 17 hours apart, about 16 hours apart, about 15 hours apart, about 14 hours apart, about 13 hours apart, about 12 hours apart, about 11 hours apart, about 10 hours apart, about 9 hours apart, about 8 hours apart, about 7 hours apart, about 6 hours apart, about 5 hours apart, about 4 hours apart, about 3 hours apart, about 2 hours apart, about 1 hour apart, about 55 minutes apart, about 50 minutes apart, about 45 minutes apart, about 40 minutes apart, about 35 minutes apart, about 30 minutes apart, about 25 minutes apart, about 20 minutes apart, about 15 minutes apart, about 10 minutes apart, or about 5 minutes apart. In other embodiments, (i) cytotoxic fusion protein and (ii) PD-1 pathway inhibitor(s) can each be administered by the same or different dosing regimen, e.g., independently selected from once daily, twice daily, three times daily, four times daily, 6 times days, 8 times daily, once weekly twice weekly, three times weekly, 4 times weekly or by continuous administration (e.g., by infusion or depot) for a period of 30 minutes to about 48 hours. In certain embodiments, the cytotoxic fusion protein and the PD-1 pathway inhibitor(s) are administered to the subject simultaneously or substantially simultaneously. In certain of these embodiments, (i) the cytotoxic fusion protein and (ii) the PD-1 pathway inhibitor(s) disclosed herein may be administered as part of a single formulation. Included are kits where (i) one or more cytotoxic fusion proteins and (ii) one or more PD-1 pathway inhibitor(s) described herein are contained within a kit together, for example, as a co-packaging arrangement.

Also contemplated herein is any variation of the above with respect to the sequence of administering (i) the cytotoxic fusion protein and (ii) PD-1 pathway inhibitor in combination. In some embodiments, the cytotoxic fusion protein is not administered prior to the PD-1 pathway inhibitor. In other embodiments, the cytotoxic fusion protein is administered prior to the PD-1 pathway inhibitor.

As another non-limiting example, the (i) cytotoxic fusion protein and/or the (ii) PD-1 pathway inhibitors and one or more additional therapeutic agents (e.g., one or more additional immune checkpoint inhibitors) can be administered about a week apart, about 6 days apart, about 5 days apart, about 4 days apart, about 3 days apart, about 2 days apart, about 24 hours apart, about 23 hours apart, about 22 hours apart, about 21 hours apart, about 20 hours apart, about 19 hours apart, about 18 hours apart, about 17 hours apart, about 16 hours apart, about 15 hours apart, about 14 hours apart, about 13 hours apart, about 12 hours apart, about 11 hours apart, about 10 hours apart, about 9 hours apart, about 8 hours apart, about 7 hours apart, about 6 hours apart, about 5 hours apart, about 4 hours apart, about 3 hours apart, about 2 hours apart, about 1 hour apart, about 55 minutes apart, about 50 minutes apart, about 45 minutes apart, about 40 minutes apart, about 35 minutes apart, about 30 minutes apart, about 25 minutes apart, about 20 minutes apart, about 15 minutes apart, about 10 minutes apart, or about 5 minutes apart. In certain embodiments, (i) one or more cytotoxic fusion proteins, (ii) one or PD-1 pathway inhibitors, and/or (iii) one or more additional therapeutic agents (e.g., one or more additional immune checkpoint inhibitors) are administered to the subject simultaneously or substantially simultaneously. In certain of these embodiments, (i) one or more cytotoxic fusion proteins, (ii) one or more PD-1 pathway inhibitors, and/or (iii) one or more additional therapeutic agents disclosed herein (e.g., one or more additional immune checkpoint inhibitors) may be administered as part of a single formulation. Included are kits where (i) one or more chimeric fusion proteins, (ii) one or more PD-1 pathway inhibitors, and (iii) one or more additional therapeutic agents (e.g., one or more additional immune checkpoint inhibitors) are contained within a kit together, for example as a co-packaging arrangement.

Also contemplated herein is any variation of the above with respect to the sequence of administering (i) one or more chimeric fusion proteins, (ii) one or more PD-1 pathway inhibitors, and (iii) one or more additional therapeutic agents (e.g., one or more additional immune checkpoint inhibitors) in combination.

The therapeutic agents and combinations for use in the methods described herein can be formulated according to any available conventional method. Examples of dosage forms include an implant, an infusion, an injectable, and the like. In the formulation, generally used additives such as a diluent, a binder, a disintegrant, a lubricant, and if necessary, a stabilizer, an emulsifier, an absorption enhancer, a surfactant, a pH adjuster, an antiseptic, an antioxidant, and the like can be used. Suitable excipients include, e.g., sugars, polyols, amino acids, surfactants, and polymers (see, e.g., Wang et al., “Antibody Structure, Instability, and Formulation,” J. Pharm. Sci. 96(1):1-26 (2007), which is hereby incorporated by reference in its entirety). In addition, the formulation is also carried out by combining compositions that are generally used as a raw material for the pharmaceutical formulation, according to conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicate and aluminum silicate; (13) purified water, and the like.

Additives for use in the above formulations may include, for example, (1) lactose, corn starch, sucrose, glucose, mannitol, sorbitol, crystalline cellulose and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; (3) starch, agar, gelatine powder, crystalline cellulose, calcium carbonate, sodium bicarbonate, calcium citrate, dextrin, pectic, carboxymethylcellulose/calcium and the like as the disintegrant; (4) magnesium stearate, talc, polyethyleneglycol, silica, condensed plant oil and the like as the lubricant; (5) any colorants whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

The therapeutic agents and combinations for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein and mixtures comprising two or more of such compounds, pharmaceutically acceptable salts of the compounds, and pharmaceutically acceptable solvates of the compounds. Such compositions are prepared in accordance with acceptable pharmaceutical procedures such as described in REMINGTON: THE SCIENCE AND PRACTICE OF PHARMACY, 21ST EDITION, ED. DAVID B. TROY (2005), which is incorporated herein by reference in its entirety.

The term “pharmaceutically acceptable carrier” refers to a carrier that does not cause an allergic reaction or another untoward effect in patients to whom it is administered and is compatible with the other ingredients in the formulation. Pharmaceutically acceptable carriers include, for example, pharmaceutical diluents, excipients, or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives, or buffers, which enhance the shelf life or effectiveness of the therapeutic agent.

Reference to therapeutic agents described herein includes any analog, derivative, isomer, metabolite, pharmaceutically acceptable salt, pharmaceutical product, hydrate, N-oxide, crystal, polymorph, prodrug, or any combination thereof.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide, and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt, and the like are preferred as “pharmacologically acceptable salt” of the compounds disclosed herein. The present invention also contemplates hydrates and solvates thereof.

In certain embodiments, the therapeutic agents disclosed herein may be in a prodrug form, meaning that it must undergo some alteration (e.g., oxidation or hydrolysis) to achieve their active form.

By way of example, suitable modes of systemic administration of the therapeutic agents and/or combinations disclosed herein include, without limitation, orally, topically, transdermally, parenterally, intradermally, intrapulmonary, intramuscularly, intraperitoneally, intravenously, intratumorally, subcutaneously, or by intranasal instillation, by intracavitary or intravesical instillation, intraocularly, intraarterially, intralesionally, or by application to mucous membranes. In certain embodiments, the therapeutic agents of the methods described herein are delivered intravenously. The different active agents can be independently administered to a selected route, e.g., to the same route or by different routes.

Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting the delivery of the agent will vary depending on the type of therapeutic agent and the cancer to be treated.

A therapeutically effective amount of a combination of therapeutic agents (e.g., one or more chimeric fusion protein(s), one or more PD-1 pathway inhibitor(s), and optionally one or more additional therapeutic agents) in the methods disclosed herein is an amount that, when administered over a particular time interval, results in the achievement of one or more therapeutic benchmarks (e.g., inhibiting the growth and/or proliferation of a target cell in a subject; slowing or halting of tumor growth, resulting in tumor regression, cessation of symptoms, etc.). The combination for use in the presently disclosed methods may be administered to a subject one time or multiple times. In those embodiments where the compounds are administered multiple times, they may be administered at a set interval, e.g., daily, every other day, weekly, or monthly. Alternatively, they can be administered at an irregular interval, for example, on an as-needed basis based on symptoms, patient health, and the like. For example, a therapeutically effective amount of a combination may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the tumor is monitored during or after the treatment, for example, by an FES-PET scan of the subject. The dosage of the combination administered to the subject can be increased or decreased depending on the status of the cancer or the regression of the tumor detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience nausea or other more serious reactions that prevent further drug administrations.

For example, a dose of 6-12 μg/kg/day of the cytotoxic fusion protein (optionally, for 5 consecutive days per 21-day cycle) may be used in the methods of the present invention. In some embodiments, a dose of 200-500 mg of the PD-1 pathway inhibitor (e.g., an anti-PD-1 antibody) may be administered every 2-4 weeks (optionally, by i.v. infusion).

In some embodiments, the PD-1 pathway inhibitor is anti-PD1-antibody nivolumab (OPDIVO®). In accordance with such embodiments, nivolumab (OPDIVO®) is administered at a dose of 240 mg every 2 weeks or 480 mg every 4 weeks until disease progression or unacceptable toxicity. Nivolumab (OPDIVO®) may be administered as a 30-minute IV infusion.

In other embodiments, the PD-1 pathway inhibitor is anti-PD1-antibody pembrolizumab (KEYTRUDA®). In accordance with such embodiments, pembrolizumab (KEYTRUDA®) is administered at a dose of 200 mg for adults and 2 mg/kg up to a maximum of 200 mg for pediatric patients. Pembrolizumab (KEYTRUDA®) may be administered as an IV infusion over 30 minutes every 3 weeks.

In further embodiments, the PD-1 pathway inhibitor is anti-PDL1-antibody atezolizumab (TECENTRIQ®). In accordance with such embodiments, atezolizumab (TECENTRIQ®) is administered at a dose of 1200 mg in combination with carboplatin AUC 5 mg/ml/min on day 1 and etoposide at a dose of 100 mg/m² on days 1-3. For maintenance, atezolizumab (TECENTRIQ®) may be administered at a dose of 840 mg every 2 weeks, 1200 mg every three weeks, or 1680 mg every four weeks until disease progression or unacceptable toxicity.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

As used herein, the term “treating” includes treating, preventing, reducing the incidence of, ameliorating symptoms of, or providing a therapeutic benefit, and, in the context of cancer, includes reducing, preventing, or inhibiting tumor cell proliferation or killing of tumor or cancer cells, reducing tumor size, inhibiting or preventing metastasis and/or the invasiveness of a tumor, and preventing the spread or recurrence of a tumor or cancer.

The effectiveness of the methods of the present application in treating the cancer in the subject may be evaluated, for example, by assessing changes in tumor burden and/or disease progression following treatment with the one or more therapeutic agents described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, tumor burden and/or disease progression is evaluated using imaging techniques including, e.g., X-ray, computed tomography (CT) scan, magnetic resonance imaging, mammography, and/or ultrasound (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Tumor burden and/or disease progression may be monitored prior to, during, and/or following treatment with one or more of the therapeutic agents described herein.

When the cancer is lymphoma, the effectiveness of the methods of the present application in treating the lymphoma in a subject may be evaluated, for example, according to the Lymphoma Response to Immunomodulatory Therapy Criteria (LYRIC) (Cheson et al., “Refinement of the Lugano Classification Lymphoma Response Criteria in the Era of Immunomodulatory Therapy,” Blood 128(21):2489-2496 (2016), which is hereby incorporated by reference in its entirety). In some embodiments, tumor burden/disease progression is evaluated by observing, e.g., changes in overall tumor burden and the appearance of new lesions or growth of one or more existing lesions using imaging techniques.

In some embodiments, the response to treatment with the methods described herein results in at least about 1%, 2%, 3%, 4%, 5%, 6%, 7%, 8%, 9%, 10%, 20%, 30%, 40%, 50%, 60%, 70%, 80%, 90%, 95%, 99%, or 100% decrease in the size of a cancer/tumor/lesion as compared to baseline cancer/tumor/lesion size. Thus, the response to treatment with any of the methods described herein may be partial (e.g., at least a 30% decrease in cancer/tumor/lesion size, as compared to baseline cancer/tumor/lesion size) or complete (elimination of a cancer/tumor/lesion).

In some embodiments, the methods described herein may be effective in inhibiting disease progression.

According to some embodiments, administering one or more of the therapeutic agents (i.e., the chimeric fusion protein and/or the PD-1 pathway inhibitor of the present disclosure) is effective to reduce at least one symptom of a disease or condition that is associated with a cancer in a subject. For example, administering the one or more of the therapeutic agents described herein may be effective to decrease a symptom of the disease or condition associated with cancer (e.g., the size or a primary tumor, the presence of metastasis, the size of a metastasis) in a subject by at least 5%, 10%, 15%, 20%, 25%, 30%, 35%, 40%, 45%, 50%, 55%, 60%, 65%, 70%, 75%, 80%, 85%, 90%, 95%, 96%, 98%, 99%, or 100%. In other embodiments, the administering is effective to mediate an improvement in the disease or condition that is associated with a cancer in a subject. In further embodiments, the administering is effective to prolong survival in the subject as compared to expected survival if no administering were carried out.

In some embodiments, administering the recombinant fusion protein in combination with the PD-1 pathway inhibitor inhibits the growth and/or proliferation of cancer cells in a subject to a greater extent than when the subject is administered the cytotoxic fusion protein or PD-1 inhibitor alone.

In some embodiments, administering the recombinant fusion protein in combination with the PD-1 pathway inhibitor has an additive effect on inhibiting cancer cell growth and/or proliferation as compared to when the subject is administered the cytotoxic fusion protein or PD-1 inhibitor alone.

In some embodiments, when the subject has been previously treated with a cytotoxic fusion protein monotherapy, the method is effective to inhibit the growth and/or proliferation of cancer cells in the subject to a greater extent than when the subject is treated with the cytotoxic fusion protein monotherapy.

In other embodiments, when the subject has been previously treated with a PD-1 pathway inhibitor monotherapy, the method is effective to inhibit the growth and/or proliferation of cancer cells in the subject to a greater extent than when the subject is treated with the PD-1 pathway inhibitor monotherapy.

The methods described herein are effective in inhibiting the growth and/or proliferation of cancer cells in the subject to a greater extent than the sum of the individual effects of (i) administering the PD-1 pathway inhibitor alone and (ii) administering the cytotoxic fusion protein alone. Thus, the methods described herein provide a synergistic effect.

As used herein, the term “survival” refers to a living patient and includes overall survival as well as progression-free survival. One-year and two-year survival rates refer to estimates of the proportion of subjects alive at 12 or 24 months. The term “overall survival” refers to the time from the start of treatment that the patient remains alive. The term “progression-free survival” refers to the time from treatment to the first day of disease progression.

The term “prolonging survival” refers to an increase in overall survival/or progression-free survival in treated patients as compared to a control treatment protocol such as treatment with a cytotoxic fusion protein monotherapy or a PD-1 pathway inhibitor monotherapy. Survival may be at least about one month, two months, three months, four months, five months, six months, 7 months, eight months, nine months, 10 months, 11 months, or at least about one year, at least about two years, at least about 3 years, at least about 4 years, at least about 5 years, at least about 6 years, at least about 7 years, at least about 8 years, at least about 9 years, at least about 10 years, or more after initiation of treatment or after initial diagnosis. It is monitored such as a year, or at least about four years, or at least about five years, or at least about ten years.

In some embodiments, said administering to the subject a composition comprising the cytotoxic fusion protein and said administering to the subject the PD-1 pathway inhibitor is effective to prolong the survival (i.e., overall survival and/or progression-free survival) of the selected subject to a greater extent than when the selected subject is treated with the cytotoxic fusion monotherapy.

In some embodiment, said administering to the subject a composition comprising the cytotoxic fusion protein and said administering to the subject the PD-1 pathway inhibitor is effective to prolong the survival (i.e., overall survival and/or progression-free survival) of the selected subject to a greater extent than when the selected subject is treated with the PD-1 pathway inhibitor monotherapy.

In some embodiments, where the subject has been previously treated with cytotoxic fusion protein monotherapy, said administering to the subject a composition comprising the cytotoxic fusion protein and said administering to the subject the PD-1 pathway inhibitor is effective to prolong the survival (i.e., overall survival and/or progression-free survival) of the selected subject to a greater extent than when the selected subject was treated with the cytotoxic fusion monotherapy.

In some embodiments, where the subject has been previously treated with a PD-1 pathway inhibitor monotherapy, said administering to the subject a composition comprising the cytotoxic fusion protein and said administering to the subject the PD-1 pathway inhibitor is effective to prolong the survival (i.e., overall survival and/or progression-free survival) of the selected subject to a greater extent than when the selected subject was treated with the PD-1 pathway inhibitor monotherapy.

In other embodiments, said administering to the selected subject a composition comprising the cytotoxic fusion protein and said administering to the subject the PD-1 pathway inhibitor is effective to prolong the survival (i.e., overall survival and/or progression-free survival) of the selected subject to a greater extent that the sum of the individual effects of (i) treating the selected subject with the cytotoxic fusion protein monotherapy and (ii) treating the selected subject with the PD-1 pathway monotherapy.

In some embodiments, administering is effective to enhance the immune response to a cancer cell population in the subject. In this context, the administering step is effective in increasing the activity of cytotoxic T cells in the subject being treated (e.g., increased production of cytotoxic cytokines (e.g., IFNγ or TNFα) and/or increase antigen-specific immune response by increasing T cell proliferation or increasing viral clearance.

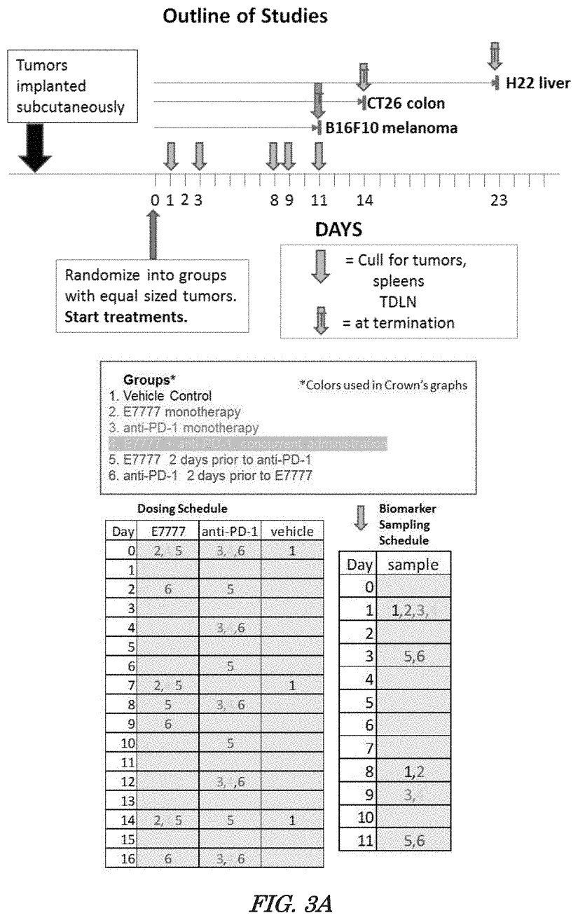

As illustrated in FIG. 2, the high-affinity human IL-2R comprises three membrane proteins: the 55 kD IL-2Rα chain (TAC, CD25), the 70-75 kD IL-2Rβ chain (CD122), and the 64 kD IL-2Rγ chain (CD132). After binding to the IL-2 receptor on the cell surface, the cytotoxic fusion protein is internalized by receptor-mediated endocytosis. The fusion protein is subsequently cleaved, releasing the N-terminus (i.e., the diphtheria toxin enzymatic and translocation domains) from the C-terminus (i.e., human IL-2), resulting in the inhibition of protein synthesis and ultimately, cell death. The monomeric species of denileukin diftitox specifically binds to the CD25 component of the high-affinity IL-2R on target cells (e.g., cancer/tumor cells); following internalization of the fusion protein, the diphtheria toxin inhibits protein synthesis.

In some embodiments, the administering is effective in inhibiting the growth and/or proliferation of cancer cells expressing the CD25 component of the IL-2 receptor. In other embodiments, the administering is effective to induce cell death in malignant cells expressing the CD25 component of the IL-2 receptor. In some embodiments, the subject has a tumor, and said administering is effective to inhibit the growth and/or proliferation of tumor-infiltrating CD25⁺ cells and/or CD25⁺ tumor cells in the subject.

In some embodiments, the administering is effective in reducing at least one symptom of a disease or condition that is associated with the cancer in a subject. In other embodiments, the administering is effective to mediate an improvement in the disease or condition that is associated with the cancer in a subject. In further embodiments, the administering is effective to prolong survival in the subject as compared to expected survival if no administering were carried out.

As described herein, it has unexpectedly been found that administering a cytotoxic fusion protein can sensitize a target cell population to treatment with a PD-1 pathway inhibitor. Thus, another aspect of the technology described herein relates to a method of sensitizing a target cell population to treatment with a PD-1 pathway inhibitor. This method involves (i) selecting a target cell population and (ii) administering to the selected target cell population a composition comprising a monomeric cytotoxic fusion protein comprising an N-terminus coupled to a C-terminus, where the N-terminus comprises diphtheria toxin fragments A and B and the C-terminus comprises human IL-2, where at least 95.0% of the total cytotoxic fusion protein content of the composition is a monomeric cytotoxic fusion protein, and where said administering is effective to sensitize the target cell population to treatment with the PD-1 pathway inhibitor. The human IL-2 may comprise a receptor-binding domain of human IL-2. In some embodiments, the human IL-2 is full-length human IL-2.

According to this aspect of the technology, administering a chimeric fusion protein in combination with a PD-1 pathway inhibitor to a target cell population increases the effectiveness of the PD-1 pathway inhibitor in reducing, inhibiting, and/or suppressing the growth of the target cell population, as compared to when the PD-1 pathway inhibitor is administered as a monotherapy. Likewise, administering a chimeric fusion protein in combination with a PD-1 pathway inhibitor increases the effectiveness of the chimeric fusion protein in reducing, inhibiting, and/or suppressing the growth of the target cell population, as compared to when the chimeric fusion protein is administered as a monotherapy.

In some embodiments, the combination therapy exhibits a tumor growth inhibition (TGI) of from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 50% to about 95%, from about 55% to about 95%, from about 60% to about 95%, from about 65% to about 95%, from about 70% to about 95%, from about 75% to about 95%, from about 80% to about 95%, from about 85% to about 95%, from about 90% to about 95%, from about 50% to about 90%, from about 55% to about 90%, from about 60% to about 90%, from about 65% to about 90%, from about 70% to about 90%, from about 75% to about 90%, from about 80% to about 90%, from about 85% to about 90%, from about 50% to about 85%, from about 55% to about 85%, from about 60% to about 85%, from about 65% to about 85%, from about 70% to about 85%, from about 75% to about 85%, from about 80% to about 85%, from about 50% to about 80%, from about 55% to about 80%, from about 60% to about 80%, from about 65% to about 80%, from about 70% to about 80%, from about 75% to about 80%, or any amount there between.

In some embodiments, the combination therapy exhibits a TGP % of from about 80% to about 95% after administration for a time point of 7 days, 14 days, 23 days or 30 days, or any amount of time therebetween. In such embodiments, the TGI % can be exhibited by, e.g., liver or colon tumors.

In some embodiments, the patient administered the combination therapy may exhibit a weight loss of less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or 0%.

In other embodiments, the patient administered the combination therapy for 7 days, 30 days, 60 days or 90 days exhibits a weight loss of less than 15%, less than 10%, less than 5% or less than 1% or maintains or increases weight after the time period.

In some embodiments, the target cells comprise T cells. As used herein, “T cells” refers to a subpopulation of lymphocytes that mature in the thymus, and which display, among other molecules T cell receptors on their surface. T cells can be identified by virtue of certain characteristics and biological properties, such as the expression of specific surface antigens including the T cell receptor (TCR), CD4, CD8, and/or CD25; the ability of certain T cells to kill tumor or infected cells; the ability of certain T cells to activate other cells of the immune system; and the ability to release protein molecules called cytokines that stimulate or inhibit the immune response. Any of these characteristics and activities can be used to identify T cells, using methods well known in the art.

In some embodiments, the target cells comprise T regulatory (Treg) cells. As used herein, the terms “T regulatory cell” or “Treg cell” or “Tregs” refers to a specialized subset of CD4⁺ T cells that functions in the establishment and maintenance of immune tolerance by suppressing conventional T cells, B cells, natural killer (NK) cells, dendritic cells (DC), and macrophages. In some embodiments, the Tregs are tumor-infiltrating Tregs. The Tregs may be CD4⁺CD25⁺FoxP3⁺ (see, e.g., Zhao et al., “Tregs: Where We Are and What Comes Next?,” Front. Immunol. 8: 1578 (2017), which is hereby incorporated by reference in its entirety).

In some embodiments, the target cells are CD25⁺ cells.

The target cells may be a population of human cells.

In some embodiments, the target cells are cancer cells, e.g., lymphoma cells.

Suitable cancers are described in more detail above and include, e.g., a lymphoma. For example, the lymphoma may be a cutaneous T-cell lymphoma (CTCL) or a peripheral T-cell lymphoma (PTCL). The CTCL may be selected from the group consisting of mycosis fungoides (MF), Sézary syndrome (SS), granulomatous slack skin (GSS), lymphomatoid papulosis (LyP), pagetoid reticulosis (PR), primary cutaneous anaplastic large cell lymphomas (PCALCL), and subcutaneous panniculitis T-cell lymphoma (SPTCL). The PTCL may be selected from the group consisting of peripheral T-cell lymphoma not otherwise specified (PTCL-NOS), angioimmunoblastic T-cell lymphoma (AITL), systemic anaplastic large cell lymphoma-anaplastic lymphoma kinase positive (sALCL-ALK⁺), systemic anaplastic large cell lymphoma-anaplastic lymphoma kinase negative (sALCL-ALK⁻), adult T-cell leukemia/lymphoma (ATLL), and enteropathy-associated T-cell lymphoma (EATL).

In other embodiments, the cancer is selected from the group consisting of breast cancer, uterine corpus cancer, cervical cancer, ovarian cancer, prostate cancer, lung cancer, stomach cancer, non-small cell lung cancer, spleen cancer, head and neck squamous cell carcinoma, esophageal cancer, bladder cancer, melanoma, colorectal cancer, kidney cancer, non-Hodgkin lymphoma, urothelial cancer, sarcoma, blood cell carcinoma, bile duct carcinoma, gallbladder carcinoma, thyroid carcinoma, prostate cancer, testicular carcinoma, thymic carcinoma, and hepatocarcinoma. In some embodiments, the cancer is not melanoma.

In some embodiments, the target cell population exhibits resistance to treatment with PD-1 inhibitor monotherapy prior to said administering.

The term “sensitize” is a relative term that refers to an increase in the degree of effectiveness of a therapeutic agent (e.g., the PD-1 pathway inhibitors described herein) in reducing, inhibiting, suppressing growth, or killing of a target cell and/or target cell population (e.g., a cancer cell, a CD25-positive cell, and/or a T regulatory cell). The term “growth” as used herein, encompasses any aspect of the growth, proliferation, and progression of a target cell (e.g., cancer cells, CD25-positive cells, and/or T regulatory cells), including, e.g., viability, cell division (i.e., mitosis), cell growth (e.g., increase in cell size), an increase in genetic material (e.g., prior to cell division), and metastasis. Reduction, inhibition, and/or suppression of target cell growth includes, but is not limited to, inhibition of target cell growth as compared to the growth of untreated or mock-treated target cells, reduction in cell viability, inhibition of proliferation, inhibition of metastases, induction of target cell senescence, induction of target cell death, and/or reduction of target tumor/cancer cell size. An increase in sensitivity to therapy may be measured by, e.g., using cell proliferation assays and/or cell cycle analysis assays.

In some embodiments, the a target cell population is sensitized to treatment with one or more PD-1 pathway inhibitors by at least ˜1% (e.g., at least about 1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least ˜50%, at least ˜60%, at least ˜70%, at least ˜80%, at least ˜90%, at least ˜95%, at least ˜99%, ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜100%) as compared to when the target cell population is not treated with the cytotoxic fusion protein described herein. For example, treatment of a target cell population according to the methods described herein may be effective to decrease the viability or inhibit the proliferation of the target cell population by at least 10%, at least 20%, at least 30%, at least 40%, at least 50%, at least 60%, at least 70%, at least 80%, at least 90%, or more following administration of a PD-1-pathway inhibitor, as compared to when the target cell population is not treated with a cytotoxic fusion protein described herein.

In some embodiments, the target cell population is sensitized to treatment with one or more PD-1-pathway inhibitors within a range having a lower limit selected from ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, and ˜99%, and an upper limit selected from ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, and ˜100%, or any combination thereof. For example, treatment of the target cell population according to the methods described herein may be effective to decrease the viability or inhibit the proliferation of the target cell population by 70% to 90% following administration of a cytotoxic fusion protein described herein, as compared to when the target cell population is not treated with the cytotoxic fusion protein according to the methods described herein.

In some embodiments of the methods described herein, the cytotoxic fusion protein comprises the amino acid sequence of SEQ ID NO:1.

As described herein above, the PD-1 pathway inhibitor may be an anti-PD-1 antibody selected from the group consisting of nivolumab (OPDIVO®), pembrolizumab (KEYTRUDA®), cemiplimab (LIBTAYO®), pidilizumab (CT-011), REGN2810 (SAR-439684), spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514 (MEDI0680), and PF-06801591.

The method for sensitizing a target cell population to treatment with a PD-1 pathway inhibitor may be carried out in vitro or in vivo.

In some embodiments, selecting a target cell population involves selecting a subject having a CD25⁺ lymphoma or a CD25⁺ tumor and said administering is to the selected subject. In accordance with such embodiments, said administering the cytotoxic fusion protein may be carried out at a dose of 6-12 μg/kg/day.

As described in more detail above, administering the recombinant cytotoxic fusion protein may be carried out orally, topically, transdermally, parenterally, intradermally, intrapulmonary, intramuscularly, intraperitoneally, intravenously, intratumorally, subcutaneously, or by intranasal instillation, by intracavitary or intravesical instillation, intraocularly, intraarterially, intralesionally, or by application to mucous membranes.

The method for sensitizing a target cell population to treatment with a PD-1 pathway inhibitor described herein may further involve administering the PD-1 pathway inhibitor to the selected cells. In accordance with such embodiments, administering the recombinant cytotoxic fusion protein may be carried out before, after, or simultaneously with the PD-1 pathway inhibitor. In some embodiments, administering the composition comprising a monomeric cytotoxic fusion protein in combination with administering the PD-1 pathway inhibitor is effective to increase the proportion of CD8⁺ cells in the target cell population (e.g., a tumor cell population) relative to when the target cell population is administered a PD-1 monotherapy.

Another aspect is directed to compositions and kits for use in treating a subject having cancer, the composition or kit comprising (i) a monomeric cytotoxic fusion protein comprising an N-terminus coupled to a C-terminus, where the N-terminus comprises diphtheria toxin fragments A and B and the binding domain at the C-terminus comprises human IL-2, and where at least 95.0% of the total cytotoxic fusion protein content of the composition is the monomeric cytotoxic fusion protein; and (ii) a programmed cell death-1 receptor (PD-1) pathway inhibitor. In some embodiments, the human IL-2 comprises the receptor-binding domain of human IL-2. In some embodiments, the human IL-2 comprises full-length IL-2. In some embodiments, the human IL-2 consists of full-length IL-2.

Suitable monomeric cytotoxic fusion proteins and PD-1 pathway inhibitors are described in detail above.

### EXAMPLES

Examples are provided below to illustrate the present invention. These examples are not meant to constrain the present invention to any particular application or theory of operation.

### Materials and Methods for Examples 1-3

### Animals

For H22 murine liver syngeneic model and CT26 murine colorectal syngeneic model studies, female BALB/C mice (Mus musculus) were ordered from Shanghai Lingchang Biotechnology Co., Ltd (Shanghai, China). Mice were aged 6-8 weeks (at inoculation). Mice were estimated to have a bodyweight>17 g at study initiation.

For B16F10 murine melanoma syngeneic model studies, female C57BL/6 mice (Mus musculus) were ordered from Shanghai Lingchang Biotechnology Co., Ltd (Shanghai, China). Mice were aged 6-8 weeks (at inoculation). Mice were estimated to have a body weight>17 g at study initiation

### Animal Housing

Animals were housed in polysulfone IVC cage (325 mm×210 mm×180 mm) at a density of up to 5 mice per cage. Temperature: 20-26° C. Humidity: 40-70%. Light cycle: 12 hour light (7:00 am.-7:00 pm) and 12 hours dark. Bedding material: crushed corncob bedding, autoclaved; changed weekly. Diet: standard rodent chow, irradiated, ad libitum. Water: 0.2 μm filtered, reverse osmosis (RO) water, autoclaved.

### Test and Control Therapeutic Agents

Table 1 provides the descriptions of the test and control therapeutic agents.

TABLE 1
Test and Control Therapeutic Agents

| Parameters | Descriptions | Descriptions |
|---|---|---|
| Product Identification | E7777* | Anti-PD-1 (RMP1-14) |
| Manufacturer/Supplier | Dr. Reddy's Laboratories | BioXCell |
| State of Matter | Sterile lyophilized power | Solution |
| Concentration | 300 μg/vial | 7.18 mg/ml |
| Estimated amount of | 2.5 μg/mouse × 24 | 100 μg/mouse × 24 |
| compound requested | mice/group × 4 groups × | mice/group × 4 |
| for the study | 3 doses × (1 + 50%) = | groups × 5 doses × |
| | 1.08 mg | (1 + 30%) = 62.4 mg |

*see., e.g., Duvic et al., “A Dose Finding Lead-in Study of E7777 (Diphtheria Toxin Fragment-Interleukin-2 Fusion Protein) in Persistent or Recurrent Cutaneous T-Cell Lymphoma (CTCL),” Blood 124 (21): 3097-3097 (2014), which is hereby incorporated by reference in its entirety).

### Study Design

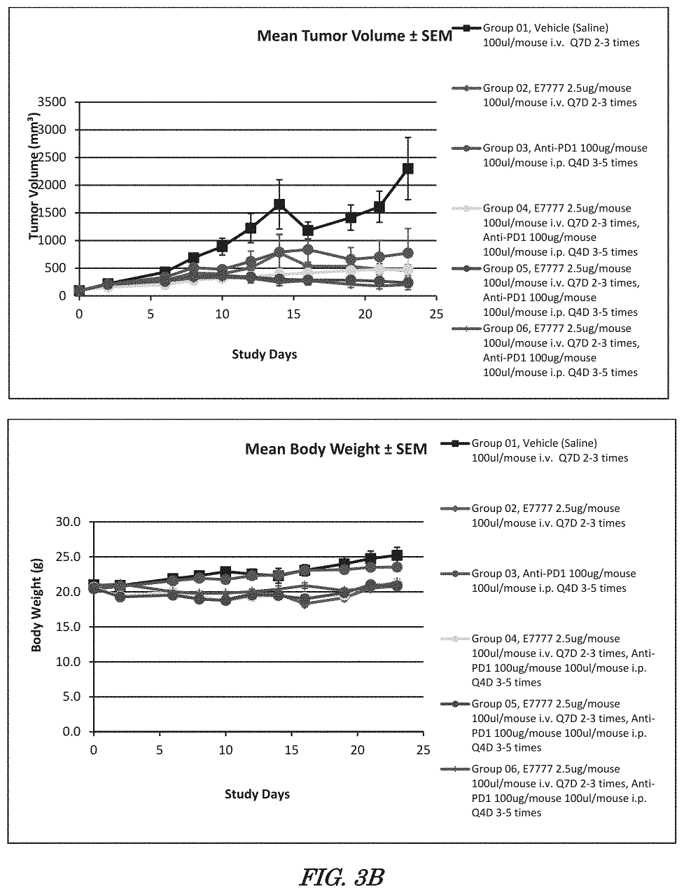

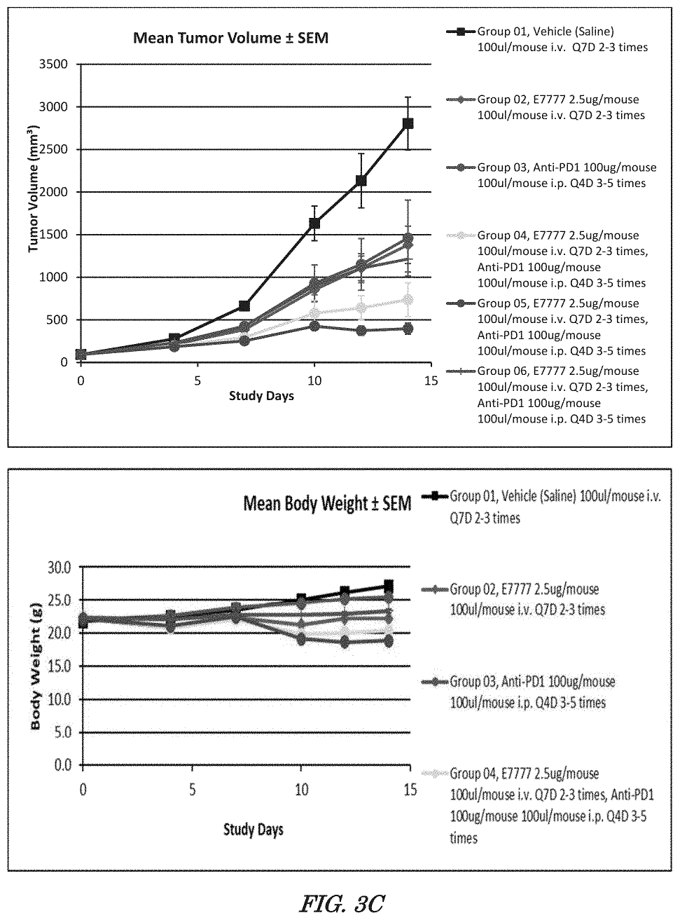

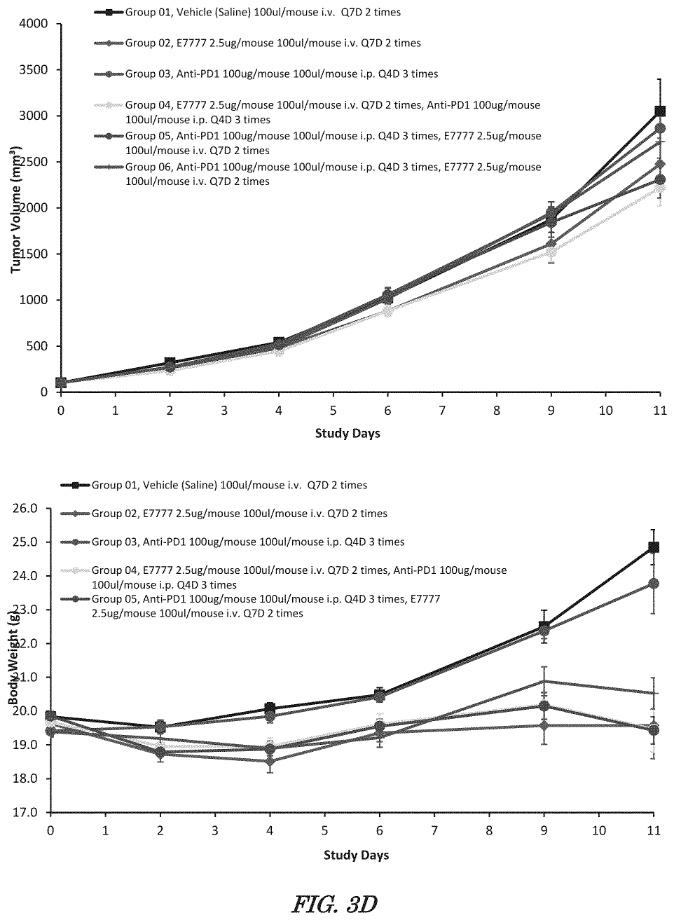

The study design is shown in FIG. 3A and Table 2 below.

TABLE 2
Study Design-H22 Murine Liver Syngeneic Model in Female BALB/c Mice,
CT26 Murine Colorectal Syngeneic Model in Female BALB/c Mice, and B16F10 Murine
Melanoma Syngeneic Model in Female C57BL/6 Mice Studies.

| | | | | Dosing | Dosing | | Dosing | |
|---|---|---|---|---|---|---|---|---|
| | | | Dose level | Solution | Volume | | Frequency | Initial Date |
| Group | N | Treatment | (μg/mouse) | (mg/ml) | (μL/mouse) | ROA | & Duration | of Dosing |
| 1 | 24 | Vehicle | — | — | 100 | i.v. | Q7D: 2-3 times | Day 0 |
| | | (Saline) | | | | | | |
| 2 | 24 | E7777 | 2.5 | 0.025 | 100 | i.v. | Q7D: 2-3 times | Day 0 |
| 3 | 24 | Anti-PD1 | 100 | 1 | 100 | i.p. | Q4D: 3-5 times | Day 0 |
| 4 | 24 | E7777 | 2.5 | 0.025 | 100 | i.v. | Q7D: 2-3 times | Day 0 |
| | | Anti-PD1 | 100 | 1 | 100 | i.p. | Q4D: 3-5 times | Day 0 |
| 5 | 24 | E7777 | 2.5 | 0.025 | 100 | i.v. | Q7D: 2-3 times | Day 0 |
| | | Anti-PD1 | 100 | 1 | 100 | i.p. | Q4D: 3-5 times | Day 2 |
| 6 | 24 | E7777 | 2.5 | 0.025 | 100 | i.v. | Q7D: 2-3 times | Day 2 |
| | | Anti-PD1 | 100 | 1 | 100 | i.p. | Q4D: 3-5 times | Day 0 |

N = animal number; ROA = route of administration; Q4D = every four days (one day dosing and 3 days off); Q7D = once a week; i.p. = intraperitoneal; i.v. = intravenous

### Cell Culture

The H22 tumor cells were maintained as a monolayer culture in RPMI-1640 medium supplemented with 10% fetal bovine serum (FBS) at 37° C. in an atmosphere with 5% CO₂. The CT26 tumor cells were maintained in vitro as a monolayer culture in RPMI-1640 medium supplemented with 10% fetal bovine serum at 37° C. in an atmosphere of 5% CO₂ in the air. The B16F10 tumor cells were maintained in vitro with 10% fetal bovine serum at 37° C. in an atmosphere of 5% CO₂ in the air. Cells in the exponential growth phase were harvested and quantitated by cell counter before tumor inoculation.

### Tumor Inoculation

Each mouse was inoculated subcutaneously in the right rear flank region with H22 (1×10⁶) in 0.1 ml of PBS, CT26 (5×10⁵) in 0.1 ml of PBS, or B16F10 (2×10⁵) in 0.1 ml of PBS for tumor development.

### Randomization

The randomization was started when the mean tumor size reached approximately 80-120 mm³. One hundred forty-four (144) mice were enrolled in the study. All animals were randomly allocated to 6 study groups. Randomization was performed based on the “Matched distribution” method using the multi-task method (StudyDirector™ software, version 3.1.399.19) randomized block design. The coefficient of variation (CV) of tumor volume in each group was calculated by the formula CV=SD/Average TV×100%. CV in each group will be lower than 30%. The date of randomization is denoted as day 0.

### Observations and Data Collection

After tumor cell inoculation, mice were checked daily for morbidity and mortality. During routine monitoring, the animals were checked for any effects of tumor growth and treatments on behavior such as, e.g., mobility, food and water consumption, body weight gain/loss (body weights were measured twice per week after randomization), eye/hair matting, and any other abnormalities.

Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured using StudyDirector™ software (version 3.1.399.19).

### Criteria for Dosing Holiday and DietGel® Administration

Body weight loss (BWL) was calculated based on the body weight (BW) of mouse on the first day of treatment. Individual mice were sacrificed after one measurement of BWL>20%. Dosing holidays were given to individual mice after one measurement of BWL>15%. Supplemental DietGel® was supplied to all the animals if >15% mean BWL is observed in the vehicle group or if >15% mean BWL is observed in the therapeutic groups.

### Study End Points

Tumor growth inhibition (TGI): TGI % is an indication of antitumor activity, and expressed as TGI (%)=100×(1−T/C). T and C are the mean tumor volume (or weight) of the treated and control groups, respectively, on a given day.

### Experimental Termination

The studies were terminated when the mean tumor burden of the vehicle-treated control group reached 2000 mm³ or one week following the final dose, whichever occurred first.

### Humane Endpoints

Body Weight Loss. The body weight of all animals was monitored throughout the study and any animals losing over 20% of their body weight relative to the weight on the first day of treatment were euthanized.

Tumor Size. Any individual mouse with a tumor volume exceeding 3000 mm³ was sacrificed. All mice in the same group were sacrificed if the mean tumor volume (MTV) of a group>2000 mm³.

Tumor Appearance Monitoring. To deter cannibalization, any animal exhibiting an ulcerated or necrotic tumor was separated immediately and singly housed and monitored daily before the animal was euthanized or until tumor regression was complete. Any animal with tumor ulceration of approximately 25% or greater on the surface of the tumor was euthanized.

General Animal Welfare Surveillance. Animals were surveilled for severe dehydration, hypothermia, abnormal/labored respiration, lethargy, obvious pain, diarrhea, skin lesions, neurological symptoms, impaired mobility (not able to eat or drink) due to significant ascites and enlarged abdomen, astasis, continuous prone or lateral position, signs of muscular atrophy, paralytic gait, clonic convulsions, tonic convulsions, persistent bleeding from body orifice.

### Terminal Sample Collection

In life, sampling was collected from tumor tissue, spleen, and the tumor-draining lymph node for evaluation by flow cytometry and immunohistochemistry (IHC), as shown in FIG. 3A.

### Statistical Analysis

For comparison between two groups, a Student's t-test was performed. All data were analyzed using SPSS 18.0 and/or GraphPad Prism 5.0. P<0.05 was considered statistically significant.

### Example 1—In Vivo Efficacy Study of Test Articles in the Treatment of 1122 Murine Liver Syngeneic Model in Female BALB/c Mice

Studies were carried out to evaluate the in vivo therapeutic efficacy of therapeutic agents in the treatment of the H22 murine liver syngeneic model in female BALB/c mice. Table 3 below shows the efficacy of the combined administration of E7777 with anti-PD-1 in terms of tumor growth inhibition (TGI). Table 4 provides a statistical analysis of tumor volume on Day 23. The results in Tables 3 and 4 demonstrate that anti-PD-1 monotherapy (Group 3) was not significantly more efficacious than vehicle control. However, all three combination therapy groups (Group 4, Group 5, and Group 6) showed significant efficacy in terms of TGI as compared to vehicle control. No difference was observed among Group 4, Group 5, and Group 6 on Day 23.

TABLE 3
H22 Liver Carcinoma Model - Tumor Growth
Inhibition with Data Collected on Day 23

| Group | Group 2 | Group 3 | Group 4 | Group 5 | Group 6 |
|---|---|---|---|---|---|
| TGI (vs vehicle, %) | 79.77 | 66.34 | 79.36 | 89.62 | 91.14 |

TABLE 4
H22 Liver Carcinoma Model - Statistical Analysis
of Tumor Volume: Comparing Tumor Volume at Day 23

| | | |
|---|---|---|
| Test | P values | Significance level |

Test of homogeneity of variance and normality

| | | |
|---|---|---|
| Bartlett's test | 1.34 × 10⁻⁵ | *** |

Test of overall equality among groups

| | | |
|---|---|---|
| Kruskal-Wallis | 0.00215 | ** |

Test of equality between individual groups

| | | |
|---|---|---|
| Group 1-Group 2 | 0.228 | ns |
| Group 1-Group 3 | 0.189 | ns |
| Group 1-Group 4 | 0.0284 | * |
| Group 1-Group 5 | 0.00155 | ** |
| Group 1-Group 6 | 0.000272 | *** |
| Group 2-Group 3 | 1 | ns |
| Group 2-Group 4 | 0.896 | ns |
| Group 2-Group 5 | 0.276 | ns |
| Group 2-Group 6 | 0.0766 | ns |
| Group 3-Group 4 | 0.933 | ns |
| Group 3-Group 5 | 0.332 | ns |
| Group 3-Group 6 | 0.0984 | ns |
| Group 4-Group 5 | 0.885 | ns |
| Group 4-Group 6 | 0.519 | ns |
| Group 5-Group 6 | 0.984 | ns |

### Example 2—In Vivo Efficacy Study of Test Articles in the Treatment of CT26 Murine Colorectal Syngeneic Model in Female BALB/c Mice

Next, the in vivo therapeutic efficacy of therapeutic agents in the treatment of the subcutaneous CT26 murine colorectal model in female BALB/c mice was evaluated. Table 5 below shows the efficacy of the combined administration of E7777 with anti-PD-1 in terms of tumor growth inhibition (TGI). Table 6 provides a statistical analysis of tumor volume on Day 14. The results in Tables 5 and 6 demonstrate that anti-PD-1 monotherapy (Group 3) was significantly more efficacious than vehicle control. Combination Group 4 and Group 5 (but not Group 6) were significantly efficacious compared to vehicle control and more significant than anti-PD-1 monotherapy. Combination Group 5 was significantly more efficacious than either anti-PD-1 or E7777 alone or combination Group 6.

TABLE 5
CT26 Colon Carcinoma Model - Tumor Growth
Inhibition with Data Collected on Day 14

| Group | Group 2 | Group 3 | Group 4 | Group 5 | Group 6 |
|---|---|---|---|---|---|
| TGI (vs vehicle, %) | 50.78 | 47.93 | 73.73 | 85.84 | 56.72 |

TABLE 6
CT26 Colon Carcinoma Model - Statistical Analysis
of Tumor Volume: Comparing Tumor Volume at Day 14

| | | |
|---|---|---|
| Test | P values | Significance level |

Test of homogeneity of variance and normality

| | | |
|---|---|---|
| Bartlett's test | 0.000589 | *** |

Test of overall equality among groups

| | | |
|---|---|---|
| Kruskal-Wallis | 0.000185 | *** |

Test of equality between individual groups

| | | |
|---|---|---|
| Group 1-Group 2 | 0.199 | ns |
| Group 1-Group 3 | 0.0456 | * |
| Group 1-Group 4 | 0.000342 | *** |
| Group 1-Group 5 | 2.74 × 10⁻⁶ | *** |
| Group 1-Group 6 | 0.072 | ns |
| Group 2-Group 3 | 0.981 | ns |
| Group 2-Group 4 | 0.153 | ns |
| Group 2-Group 5 | 0.00294 | ** |
| Group 2-Group 6 | 0.996 | ns |
| Group 3-Group 4 | 0.488 | ns |
| Group 3-Group 5 | 0.0208 | * |
| Group 3-Group 6 | 1 | ns |
| Group 4-Group 5 | 0.631 | ns |
| Group 4-Group 6 | 0.37 | ns |
| Group 5-Group 6 | 0.0122 | * |

### Example 3—In Vivo Efficacy Study of Test Articles in the Treatment of B16F10 Murine Melanoma Syngeneic Model in Female C57BL/6 Mice

The objective of this study was to evaluate preclinically the in vivo therapeutic efficacy of test articles in the treatment of the subcutaneous B16F10 murine melanoma model in female C57BL/6 mice.

The results in Table 7 demonstrate that no treatment groups were significantly efficacious as compared to vehicle control in inhibiting tumor growth in the B16F10 melanoma model.

TABLE 7
B16F10 Melanoma Model - Statistical Analysis of
Tumor Volume: Comparing Tumor Volume at Day 14

| | | |
|---|---|---|
| Test | P values | Significance level |

Test of homogeneity of variance and normality

| | | |
|---|---|---|
| Bartlett's test | 0.574 | ns |

Test of overall equality among groups

| | | |
|---|---|---|
| Kruskal-Wallis | 0.116 | ns |

Test of equality between individual groups

| | | |
|---|---|---|
| Group 1-Group 2 | 0.641 | ns |
| Group 1-Group 3 | 0.995 | ns |
| Group 1-Group 4 | 0.21 | ns |
| Group 1-Group 5 | 0.202 | ns |
| Group1-Group 6 | 0.896 | ns |
| Group 2-Group 3 | 0.901 | ns |
| Group 2-Group 4 | 0.983 | ns |
| Group 2-Group 5 | 0.996 | ns |
| Group 2-Group 6 | 0.977 | ns |
| Group 3-Group 4 | 0.48 | ns |
| Group 3-Group 5 | 0.509 | ns |
| Group 3-Group 6 | 0.997 | ns |
| Group 4-Group 5 | 1 | ns |
| Group 4-Group 6 | 0.618 | ns |
| Group 5-Group 6 | 0.648 | ns |

### Discussion of Examples 1-3

Flow cytometry and immunohistochemistry analysis were used to evaluate changes to immune cell composition in tumors, spleens, and tumor-draining lymph nodes during and after administration of E7777 and anti-mPD-1.

Flow cytometry analysis revealed that 30.5% of CD3⁺ T-cell infiltrates in H22 tumors comprised Tregs, whereas only 6.6% and 0.52% of CD3⁺ T-cell infiltrates in CT26 and B16F10 tumors comprised Tregs, respectively on day 1 (Table 8).

TABLE 8
Flow Cytometry of Tumors: Day 1, Group 1

| Flow Cytometry of Tumors: Day 1, | | | |
|---|---|---|---|
| Group 1 (N = 2/group) | H22 | CT26 | B16F10 |
| CD3⁺ T cells | 7,563 | 28,228 | 4,119 |
| FoxP3⁺ Tregs | 2304 | 1864 | 21.5 |
| Percent Tregs | 30.5% | 6.6% | 0.52% |

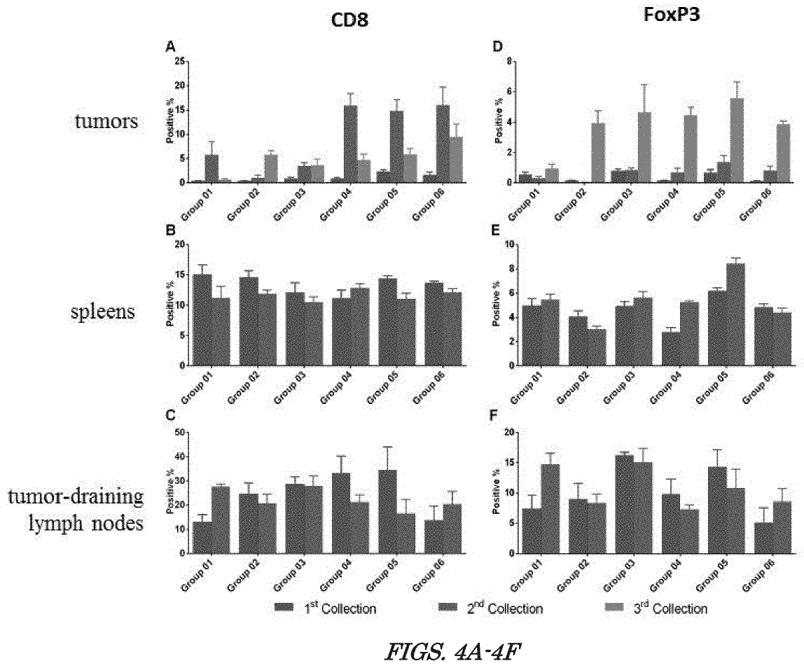

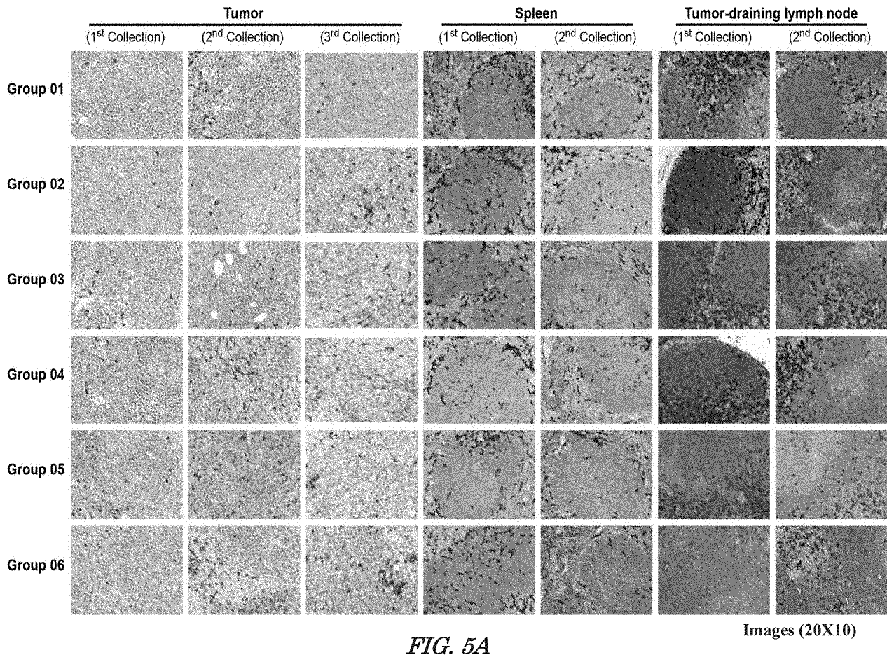

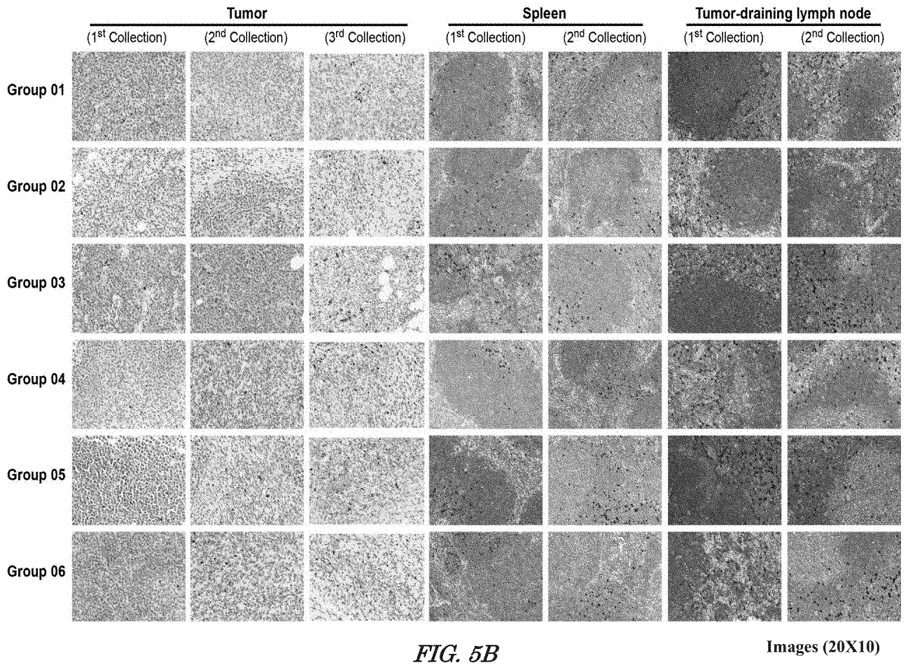

Flow cytometry and immunohistochemistry analysis of tumors, spleen, and TDLN tissues form BALB/c mice implanted with H22 cells revealed the presence of CD8⁺ and FoxP3⁺ cells in tumor, spleen, and TDLN tissues collected from Group 1, Group 2, Group 3, Group 4, Group 5, and Group 6 at various time points following treatment initiation (FIGS. 4A-4F; FIGS. 5A-5B).

Examples 1-3 above demonstrate that administering E7777 in combination with anti-PD-1 is effective in inhibiting tumor growth, as long as Treg cells are present in the tumor microenvironment.

### Materials and Methods for Examples 4-5

### Animals

For H22 murine liver syngeneic model and CT26 murine colorectal syngeneic model studies, female BALB/C mice (Mus musculus) were ordered from Shanghai Lingchang Biotechnology Co., Ltd (Shanghai, China). Mice were aged 6-8 weeks (at inoculation). Mice were estimated to have a body weight 15-20 g at study initiation.

### Animal Housing

Animals were housed in polysulfone IVC cages (325 mm×210 mm×180 mm) at a density of up to 5 mice per cage. Temperature: 20-26° C. Humidity: 40-70%. Light cycle: 12 hour light (7:00 am.-7:00 pm) and 12 hours dark. Bedding material: crushed corncob bedding, autoclaved; changed weekly. Diet: standard rodent chow, irradiated, ad libitum. Water: 0.2 μm filtered, reverse osmosis (RO) water, autoclaved.

### Test and Control Therapeutic Agents

Table 9 provides the descriptions of the test and control therapeutic agents.

TABLE 9
Test and Control Therapeutic Agents

| Parameters | Descriptions | Descriptions |
|---|---|---|
| Product Identification | E7777* | Anti-PD-1 (RMP1-14) |
| Manufacturer/Supplier | Dr. Reddy's Laboratories | CroenVivoPremium(OEM) |
| State of Matter | Sterile lyophilized power | Solution |
| Concentration | 300 μg/vial | 5.9 mg/ml |
| Estimated Amount of | 2.5 μg/mouse × 16 | 100 μg/mouse × 16 |
| Compound Requested | mice/group × 3 groups × 3 | mice/group × 3 groups × 6 |
| for the Study | doses × (1 + 50%) = 540 μg | doses × (1 + 30%) = 37.5 mg |

*see., e.g., Duvic et al., “A Dose Finding Lead-in Study of E7777 (Diphtheria Toxin Fragment-Interleukin-2 Fusion Protein) in Persistent or Recurrent Cutaneous T-Cell Lymphoma (CTCL),” Blood 124 (21): 3097-3097 (2014), which is hereby incorporated by reference in its entirety).

The study design is shown in Table 10 below.

TABLE 10
Study Design-H22 Murine Liver Syngeneic Model in Female BALB/c Mice
and CT26 Murine Colorectal Syngeneic Model in Female BALB/c Mice Tumor Growth
Inhibition and Survival Studies

| | | | | Dosing | Dosing | | Dosing | |
|---|---|---|---|---|---|---|---|---|
| | | | Dose level | Solution | Volume | | Frequency | Initial Date |
| Group | N | Treatment | (μg/mouse) | (mg/ml) | (μL/mouse) | ROA | & Duration | of Dosing |
| 1 | 16 | Vehicle | — | — | 100 | i.v. | Q7D: 3 times | Day 0 |
| | | (Saline) | | | | | | |
| 2 | 16 | E7777 | 2.5 | 0.025 | 100 | i.v. | Q7D: 3 times | Day 0 |
| 3 | 16 | Anti-PD1 | 100 | 1 | 100 | i.p. | Q4D: 6 times | Day 0 |
| 4 | 16 | E7777 | 2.5 | 0.025 | 100 | i.v. | Q7D: 3 times | Day 0 |
| | | Anti-PD1 | 100 | 1 | 100 | i.p. | Q4D: 6 times | Day 0 |
| 5 | 16 | E7777 | 2.5 | 0.025 | 100 | i.v. | Q7D: 3 times | Day 0 |
| | | Anti-PD1 | 100 | 1 | 100 | i.p. | Q4D: 6 times | Day 2 |

N = animal number; ROA = route of administration; Q4D = every four days (one day dosing and 3 days off); Q7D = once a week; i.p. = intraperitoneal; i.v. = intravenous

### Cell Culture

The H22 cell line was maintained as a monolayer culture in RPMI-1640 medium supplemented with 10% fetal bovine serum (FBS) at 37° C. in an atmosphere with 5% CO₂. The CT26 tumor cells were maintained in vitro as a monolayer culture in RPMI-1640 medium supplemented with 10% fetal bovine serum at 37° C. in an atmosphere of 5% CO₂ in the air. Cells in the exponential growth phase were harvested and quantitated by cell counter before tumor inoculation.

### Tumor Inoculation

Each mouse was inoculated subcutaneously in the right rear flank region with H22 (1×10⁶) in 0.1 ml of PBS or CT26 (5×10⁵) in 0.1 ml of PBS for tumor development.

### Randomization

Randomization was imitated when the mean tumor size reached approximately 50-100 mm³. Eighty (80) mice were enrolled in the study. All animals were randomly allocated to 5 study groups, with 16 mice per group. Tumor volume was used as a numeric parameter to randomize selected animals into specified groups. Randomization was performed based on the “Matched distribution” method using the multi-task method (StudyDirector™ software, version 3.1.399.19) randomized block design. The coefficient of variation (CV) of tumor volume in each group was calculated by the formula CV=SD/Average TV×100%. CV in each group was lower than 30%. The date of randomization was denoted as day 0.

### Observation and Data Collection

After tumor cell inoculation, mice were checked daily for morbidity and mortality. During routine monitoring, mice were checked for any effects of tumor growth and treatments on behavior such as, e.g., mobility, food and water consumption, body weight gain/loss (body weights were measured twice per week after randomization), eye/hair matting, and any other abnormalities.

Tumor volumes were measured twice per week in two dimensions using a caliper, and the volume was expressed in mm³ using the formula: “V=(L×W×W)/2, where V is tumor volume, L is tumor length (the longest tumor dimension), and W is tumor width (the longest tumor dimension perpendicular to L). Dosing as well as tumor and body weight measurements were conducted in a Laminar Flow Cabinet.

The body weights and tumor volumes were measured using StudyDirector™ software (version 3.1.399.19).

### Criteria for Dosing Holiday and DietGel® Administration

Body weight loss (BWL) was calculated based on the body weight (BW) of the mouse on the first day of treatment. Individual mice were sacrificed after one measurement of BWL>20%. Dosing holidays were given to individual mice after one measurement of BWL>15%. Treatment was resumed when the BWL recovered to <10%. Supplemental DietGel® was supplied to all the animals if >15% mean BWL is observed in the vehicle group or if >15% mean BWL is observed in the therapeutic groups.

### Study End Points

Tumor growth inhibition (TGI): TGI % is an indication of antitumor activity and expressed as TGI (%)=100χ (1−T/C). T and C are the mean tumor volume (or weight) of the treated and control groups, respectively, on a given day.

The T/C value (%) is an indicator of tumor response to treatment and one antitumor activity endpoint.

Overall survival was calculated for each group of mice.

### Study Termination

The study mice were treated and then observed, with their tumor volumes and body weights measured, for up to 60 days.

### Humane Endpoints

Body Weight Loss. The body weight of all animals was monitored throughout the study, and any animals losing over 20% of their body weight relative to the weight on the first day of treatment were euthanized.

Tumor Size. Any individual mouse with a tumor volume exceeding 3000 mm³ was sacrificed. All mice in the same group were sacrificed if the mean tumor volume (MTV) of a group was >2000 mm³.

Tumor Appearance Monitoring. To deter cannibalization, any animal exhibiting an ulcerated or necrotic tumor was separated immediately and singly housed and monitored daily before the animal was euthanized or until tumor regression was complete. Any animal with tumor ulceration of approximately 25% or greater on the surface of the tumor was euthanized.

General Animal Welfare Surveillance. Animals were surveilled for severe dehydration, hypothermia, abnormal/labored respiration, lethargy, obvious pain, diarrhea, skin lesions, neurological symptoms, impaired mobility (not able to eat or drink) due to significant ascites and enlarged abdomen, astasia, continuous prone or lateral position, signs of muscular atrophy, paralytic gait, clonic convulsions, tonic convulsions, persistent bleeding from body orifice.

### Statistical Analysis

For comparison between different groups on a pre-specified day Bartlett's test was used to check the assumption of homogeneity of variance across all groups. When the p value of Bartlett's test was ≥0.05, one-way ANOVA was run to test the overall equality of means across all groups. If the p-value of the one-way ANOVA was <0.05, post hoc testing was performed by running Tukey's HSD (honest significant difference) tests for all pairwise comparisons, and Dunnett's tests for comparing each treatment group with the vehicle group. When the p-value of Bartlett's test was <0.05, the Kruskal-Wallis test was run to test the overall equality of medians among all groups. If the p-value of the Kruskal-Wallis test was <0.05, post hoc testing was performed by running Conover's non-parametric test for all pairwise comparisons or for comparing each treatment group with the vehicle group, both with single-step p-value adjustment. In addition, pairwise comparisons were performed without multiple testing correction, and nominal/uncorrected p-values were reported directly from Welch's t-test or Mann-Whitney U test. Specifically, Bartlett's test was first used to check the assumption of homogeneity of variance for a pair of groups. When the p-value of Bartlett's test was ≥0.05, Welch's t-test was run, otherwise the Mann-Whitney U test was run to obtain nominal p-values. All statistical analyses were done in R—a language and environment for statistical computing and graphics (version 3.3.1). All tests were two-sided unless otherwise specified, and p-values of <0.05 were regarded as statistically significant.

### Example 4—Effect of Test Articles in Tumor Growth Inhibition and Survival of Syngeneic Murine Liver Cancer Model 1122 in Female BALB/c Mice

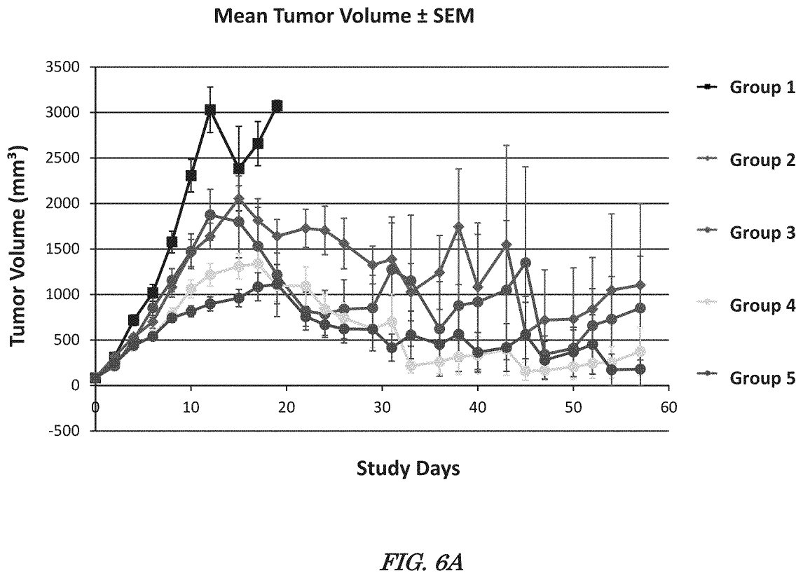

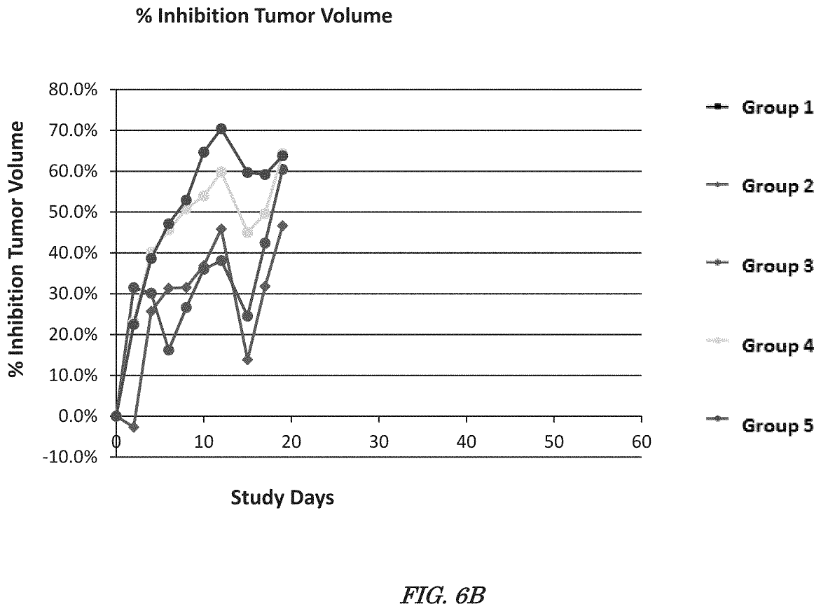

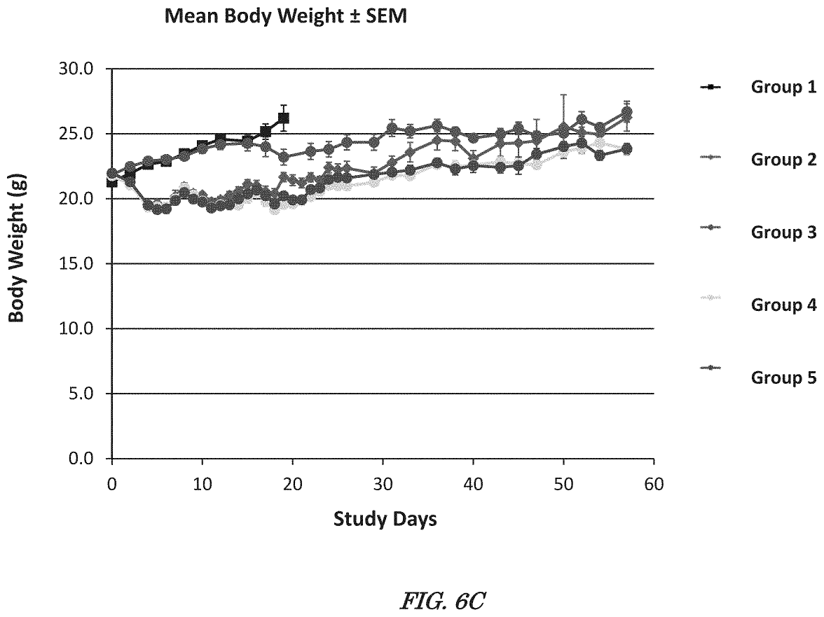

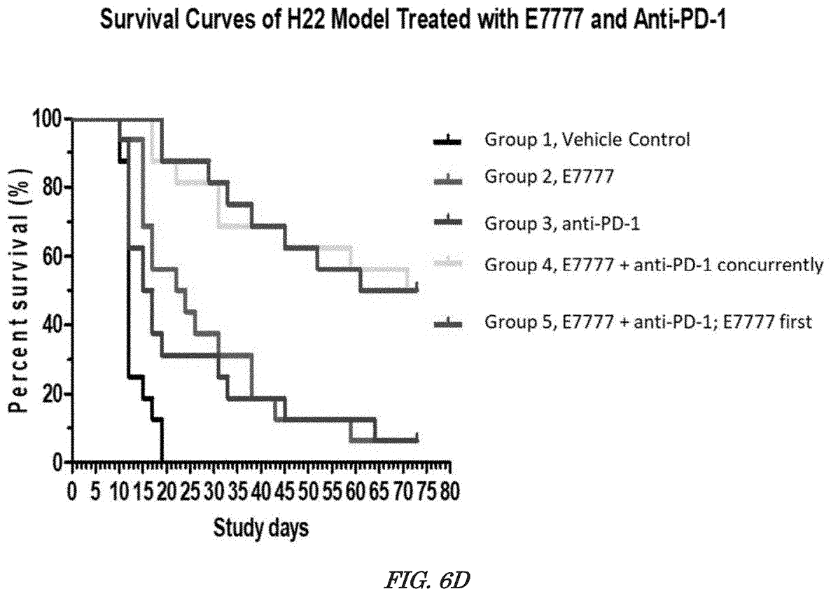

Studies were carried out to evaluate the effect of test articles in the survival study of the H22 murine liver syngeneic model in female BALB/c mice. FIGS. 6A-6B and Table 11 below show the efficacy of combined administration of E7777 with anti-PD-1 in terms of tumor growth inhibition (TGI). FIG. 6C shows the mean body weight in mice evaluated using the H22 liver carcinoma model. Table 12 provides a statistical analysis of tumor volume on Day 12. The results in FIGS. 6A-6B, Tables 11, and Table 12 demonstrate that all three combination therapy groups (Group 4 and Group 5) showed significant efficacy in terms of TGI as compared to E7777 monotherapy or anti-PD-1 monotherapy.

TABLE 11
H22 Liver Carcinoma Model - Tumor Growth
Inhibition with Data Collected on Day 12

| | | Standard | 90% CI | 90% CI |
|---|---|---|---|---|
| Group | TGI | Error | (lower bond) | (upper bond) |
| Group 2 | 47.17 | 6.606 | 35.12 | 56.92 |
| Group 3 | 39.20 | 10.459 | 20.73 | 55.25 |
| Group 4 | 61.55 | 5.283 | 51.95 | 69.55 |
| Group 5 | 72.41 | 3.476 | 66.11 | 77.48 |

TABLE 12
H22 Liver Carcinoma Model - Statistical Analysis
of Tumor Volume: Comparing Tumor Volume at Day 12

| | | |
|---|---|---|
| Test | P values | Significance level |

Test of homogeneity of variance and normality

| | | |
|---|---|---|
| Bartlett's test | 3.43 × 10⁻⁵ | *** |

Test of overall equality among groups

| | | |
|---|---|---|
| Kruskal-Wallis | 6.15 × 10⁻⁷ | *** |

Test of equality between individual groups

| | | |
|---|---|---|
| Group 1-Group 2 | 9.41 × 10⁻³ | ** |
| Group 1-Group 3 | 5.04 × 10⁻⁴ | ** |
| Group 1-Group 4 | 4.39 × 10⁻⁶ | *** |
| Group 1-Group 5 | 2.38 × 10⁻⁹ | *** |
| Group 2-Group 3 | 1.00 | ns |
| Group 2-Group 4 | 2.14 × 10⁻¹ | ns |
| Group 2-Group 5 | 1.48 × 10⁻³ | ** |
| Group 3-Group 4 | 3.11 × 10⁻¹ | ns |
| Group 3-Group 5 | 2.97 × 10⁻³ | ** |
| Group 4-Group 5 | 3.41 × 10⁻¹ | ns |

FIG. 6D and Table 13 below show the effect of combined administration of E7777 with anti-PD-1 in terms of survival at Day 73. Table 14 provides a statistical analysis of survival on Day 73. Both combination therapy groups (Group 4 and Group 5) showed significant effects in terms of survival as compared to vehicle control and as compared to either agent administered as monotherapy (Group 2 and Group 3). No significant difference was observed between Group 4 and Group 5 on Day 73.

TABLE 13
H22 Liver Carcinoma Model: Comparison of Survival at Day 73

| | Group 1 | Group 2 | Group 3 | Group 4 | Group 5 |
|---|---|---|---|---|---|
| Deaths (of 16/group) | 16 | 15 | 15 | 8 | 8 |
| Median Survival | 12 | 23 | 16 | 72 | 67 |
| (days) | | | | | |

TABLE 14
Statistical Comparisons of Survival Between Groups at Day
73 for H22 Liver Carcinoma Syngeneic Model Showing p Values

| | Group 1 | Group2 | Group 3 | Group 4 | Group 5 |
|---|---|---|---|---|---|
| Group 2 | .00027 | — | — | — | — |
| Group 3 | .01910 | .62256 | — | — | — |
| Group 4 | 1.8 × 10⁻⁷ | .00151 | .00082 | — | — |
| Group 5 | 1.4 × 10⁻⁷ | .00101 | .00068 | .95313 | — |

### Example 5—Effect of Test Articles in Tumor Growth Inhibition and Survival in Syngeneic Murine Colorectal Cancer Model CT26 in Female BALB/c Mice

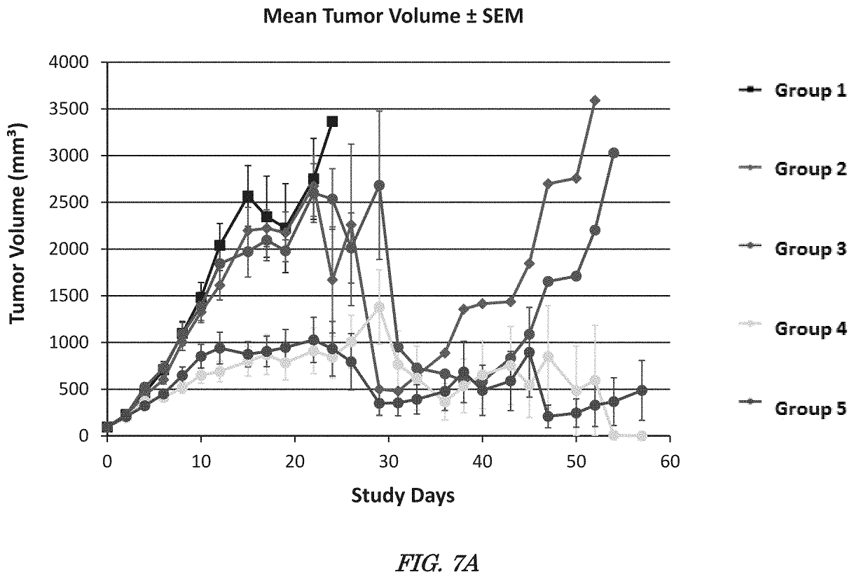

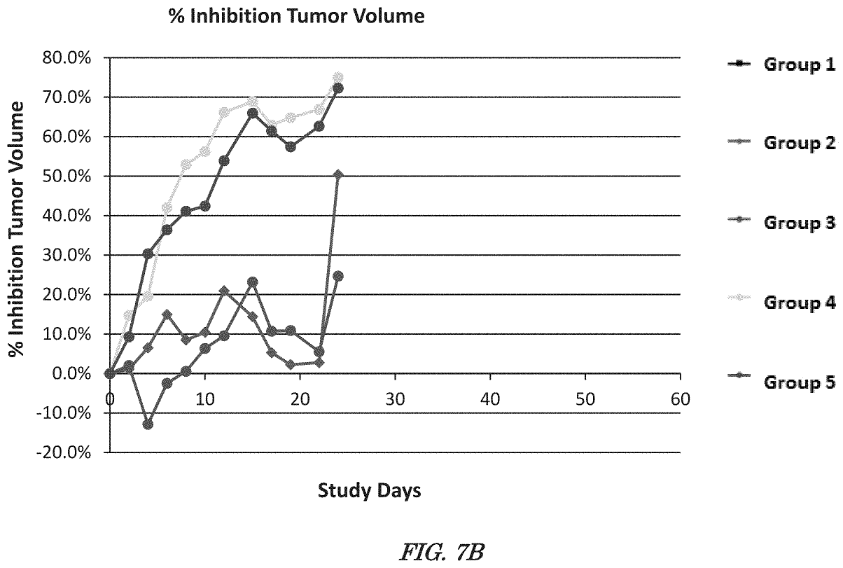

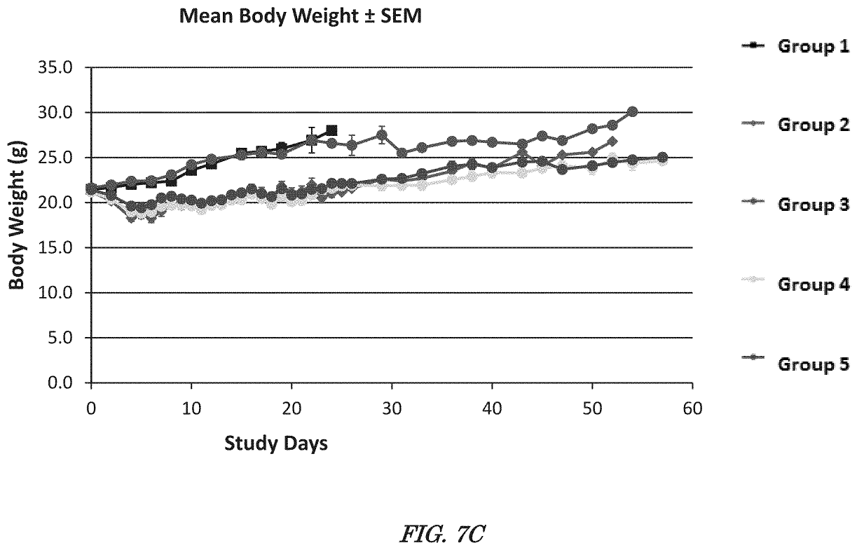

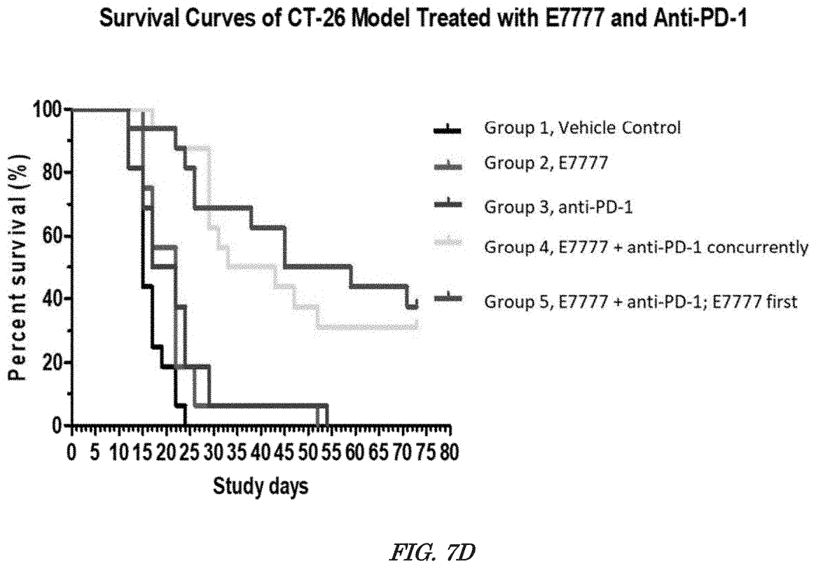

Studies were carried out to evaluate the effect of test articles in the survival study of the C26 murine colorectal syngeneic model in female BALB/c mice. FIGS. 7A-7B and Table 15 below show the efficacy of combined administration of E7777 with anti-PD-1 in terms of tumor growth inhibition (TGI). FIG. 7C shows the mean body weight in mice evaluated using the CT26 colon carcinoma model. Table 16 provides a statistical analysis of tumor volume on Day 15. The results in FIGS. 7A-7B, Table 15, and Table 16 demonstrate that combination Group 4 and Group 5 were significantly efficacious compared to anti-PD-1 monotherapy or E7777 monotherapy.

TABLE 15
CT26 Colon Carcinoma Model - Tumor Growth
Inhibition with Data Collected on Day 15

| | | Standard | 90% CI | 90% CI |
|---|---|---|---|---|
| Group | TGI | Error | (lower bond) | (upper bond) |
| Group 2 | 15.12 | 14.90 | −13.984 | 34.71 |
| Group 3 | 23.97 | 15.05 | −4.626 | 44.28 |
| Group 4 | 71.60 | 7.39 | 57.878 | 81.64 |
| Group 5 | 68.55 | 6.65 | 56.298 | 78.63 |

TABLE 16
CT26 Colon Carcinoma Model - Statistical Analysis
of Tumor Volume: Comparing Tumor Volume at Day 15

| | | |
|---|---|---|
| Test | P values | Significance level |

Test of homogeneity of variance and normality

| | | |
|---|---|---|
| Bartlett's test | 2.58 × 10⁻² | * |

Test of overall equality among groups

| | | |
|---|---|---|
| Kruskal-Wallis | 1.04 × 10⁻⁶ | *** |

Test of equality between individual groups

| | | |
|---|---|---|
| Group 1-Group 2 | 9.69 × 10⁻¹ | ns |
| Group 1-Group 3 | 6.32 × 10⁻¹ | ns |
| Group 1-Group 4 | 2.69 × 10⁻⁶ | *** |
| Group 1-Group 5 | 2.77 × 10⁻⁵ | *** |
| Group 2-Group 3 | 9.20 × 10⁻¹ | ns |
| Group 2-Group 4 | 1.06 × 10⁻⁵ | *** |
| Group 2-Group 5 | 1.15 × 10⁻⁴ | *** |
| Group 3-Group 4 | 6.44 × 10⁻⁴ | *** |
| Group 3-Group 5 | 4.37 × 10⁻³ | ** |
| Group 4-Group 5 | 9.82 × 10⁻¹ | ns |

FIG. 7D and Table 17 below show the effect of combined administration of E7777 with anti-PD-1 in terms of survival at Day 73. Table 18 provides a statistical analysis of survival on Day 73. Both combination therapy groups (Group 4 and Group 5) showed significant effects in terms of survival as compared to vehicle control and as compared to either agent administered as monotherapy (Group 2 and Group 3.) No significant difference was observed between Group 4 and Group 5 on Day 73.

TABLE 17
CT26 Colon Cancer Model: Comparison of Survival at Day 73

| | Group 1 | Group 2 | Group 3 | Group 4 | Group 5 |
|---|---|---|---|---|---|
| Deaths (of 16/group) | 16 | 16 | 16 | 11 | 10 |
| Median Survival | 15 | 22 | 19.5 | 38 | 52 |
| (days) | | | | | |

TABLE 18
Statistical Comparisons of Survival Between Groups at Day
73 for CT26 Colon Cancer Syngeneic Model Showing p Values

| | Group 1 | Group2 | Group 3 | Group 4 | Group 5 |
|---|---|---|---|---|---|
| Group 2 | .01670 | — | — | — | — |
| Group 3 | .03960 | .72768 | — | — | — |
| Group 4 | 5.1 × 10⁻⁷ | .00011 | .00016 | — | — |
| Group 5 | 1.4 × 10⁻⁶ | .00011 | .00011 | .72768 | — |

### Discussion of Examples 4-5

Examples 4-5 above demonstrate that administering E7777 in combination with anti-PD-1 synergistically increases overall survival in syngeneic models of cancer, as compared to E7777 monotherapy or anti-PD-1 monotherapy.

Although preferred embodiments have been depicted and described in detail herein, it will be apparent to those skilled in the relevant art that various modifications, additions, substitutions, and the like can be made without departing from the spirit of the invention, and these are therefore considered to be within the scope of the invention as defined in the claims which follow.

What is claimed is:

1. A method for treating a subject having a cancer, the method comprising:
administering to the subject a composition comprising a monomeric cytotoxic fusion protein comprising an N-terminus coupled to a C-terminus, wherein the N terminus comprises diphtheria toxin fragments A and B and the C-terminus comprises human IL-2, and wherein at least 95.0% of the total cytotoxic fusion protein content of the composition is a monomeric cytotoxic fusion protein; and
administering to the subject a programmed cell death-1 receptor (PD-1) pathway inhibitor to treat the cancer in the subject.

2. The method according to claim 1, wherein the subject is a human subject.

3. The method according to claim 1, wherein the subject has been previously treated with a cytotoxic fusion protein monotherapy.

4. The method according to claim 1, wherein the subject has been previously treated with a PD-1 pathway inhibitor monotherapy.

5. The method according to claim 1, wherein the cancer is selected from the group consisting of a carcinoma, a sarcoma, a leukemia, a lymphoma, and combinations thereof (mixed-type cancer).

6. The method according to claim 1, wherein the cancer is not a melanoma.

7. The method according to claim 5, wherein the cancer is a lymphoma.

8. The method according to claim 7, wherein the lymphoma is a cutaneous T-cell lymphoma (CTCL) or a peripheral T-cell lymphoma (PTCL).

9. The method according to claim 8, wherein the CTCL is selected from the group consisting of mycosis fungoides (MF), Sézary syndrome (SS), granulomatous slack skin (GSS), lymphomatoid papulosis (LyP), pagetoid reticulosis (PR), primary cutaneous anaplastic large cell lymphomas (PCALCL), and subcutaneous panniculitis T-cell lymphoma (SPTCL).

10. The method according to claim 8, wherein the PTCL is selected from the group consisting of peripheral T-cell lymphoma not otherwise specified (PTCL-NOS), angioimmunoblastic T-cell lymphoma (AITL), systemic anaplastic large cell lymphoma-anaplastic lymphoma kinase positive (sALCL-ALK⁺), systemic anaplastic large cell lymphoma-anaplastic lymphoma kinase negative (sALCL-ALK⁻), adult T-cell leukemia/lymphoma (ATLL), and enteropathy-associated T-cell lymphoma (EATL).

11. The method according to claim 1, wherein the cancer is a PD-L1 positive (PD-L1⁺) cancer.

12. The method according to claim 1, wherein the cytotoxic fusion protein comprises the amino acid sequence of SEQ ID NO:1.

13. The method according to claim 1, wherein the PD-1 pathway inhibitor is selected from the group consisting of anti-PD-1 antibody, anti-PD-L1 antibody, anti-PD-L2 antibody, anti-PD-1 RNAi, anti-PD-L1 RNAi, anti-PD-L2RNAi, anti-PD-1 antisense RNA, anti-PD-L1 antisense RNA, anti-PD-L2 antisense RNA, dominant negative PD-1 protein, dominant negative PD-L1 protein, and dominant negative PD-L2 protein.

14. The method according to claim 13, wherein the anti-PD-1-antibody is selected from the group consisting of nivolumab (OPDIVO®), pembrolizumab (KEYTRUDA®), cemiplimab (LIBTAYO®), pidilizumab (CT-011), REGN2810 (SAR-439684), spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514 (MEDI0680), and PF-06801591.

15. The method according to claim 13, wherein the anti-PD-L1 antibody is selected from the group consisting of atezolizumab (TECENTRIQ®), avelumab (BAVENCIO®), durvalumab (IMFINZI®), KN035, CK-301, AUNP12, CA-170, BMS-986189, MPDL3280A, and MEDI4736.

16. The method according to claim 1, wherein administering the recombinant cytotoxic fusion protein and/or the PD-1 pathway inhibitor is carried out orally, topically, transdermally, parenterally, intradermally, intrapulmonary, intramuscularly, intraperitoneally, intravenously, intratumorally, subcutaneously, or by intranasal instillation, by intracavitary or intravesical instillation, intraocularly, intraarterially, intralesionally, or by application to mucous membranes.

17. The method according to claim 1, wherein administering the recombinant fusion protein is carried out simultaneously with administering the PD-1 pathway inhibitor.

18. The method according to claim 1, wherein administering the recombinant fusion protein is carried out prior to administering the PD-1 pathway inhibitor.

19. The method according to claim 1, wherein administering the recombinant fusion protein is carried out after administering the PD-1 pathway inhibitor.

20. The method according to claim 1, wherein the method is effective to inhibit growth and/or proliferation of cancer cells expressing the CD25 component of the IL-2 receptor; induce cell death in malignant cells expressing the CD25 component of the IL-2 receptor; inhibit the growth and/or proliferation of tumor-infiltrating CD25⁺ cells and/or CD25⁺ tumor cells in the subject.

21. The method according to claim 3, wherein the method is effective to inhibit the growth and/or proliferation of cancer cells in the selected subject to a greater extent than when the selected subject is treated with the cytotoxic fusion protein monotherapy.

22. The method according to claim 4, wherein the method is effective in inhibiting the growth and/or proliferation of cancer cells in the selected subject to a greater extent than when the selected subject is treated with the PD-1 pathway inhibitor monotherapy.

23. The method according to claim 1, wherein said administering to the subject a composition comprising the cytotoxic fusion protein and said administering to the subject the PD-1 pathway inhibitor is effective to prolong the survival of the selected subject to a greater extent than when the selected subject is treated with the cytotoxic fusion monotherapy.

24. The method according to claim 1, wherein said administering to the subject a composition comprising the cytotoxic fusion protein and said administering to the subject the PD-1 pathway inhibitor is effective to prolong the survival of the selected subject to a greater extent than when the selected subject is treated with the PD-1 pathway inhibitor monotherapy.

25. The method according to claim 3, wherein said administering to the subject a composition comprising the cytotoxic fusion protein and said administering to the subject the PD-1 pathway inhibitor is effective to prolong the survival of the selected subject to a greater extent than when the selected subject was treated with the cytotoxic fusion monotherapy.

26. The method according to claim 4, wherein said administering to the subject a composition comprising the cytotoxic fusion protein and said administering to the subject the PD-1 pathway inhibitor is effective to prolong the survival of the selected subject to a greater extent than when the selected subject was treated with the PD-1 pathway inhibitor monotherapy.

27. The method according to claim 1, wherein said administering to the selected subject a composition comprising the cytotoxic fusion protein and said administering to the subject the PD-1 pathway inhibitor is effective to prolong the survival of the selected subject to a greater extent that the sum of the individual effects of (i) treating the selected subject with the cytotoxic fusion protein monotherapy and (ii) treating the selected subject with the PD-1 pathway monotherapy.

28. A method for sensitizing a target cell population to treatment with a PD-1 pathway inhibitor, the method comprising:
selecting a target cell population and
administering to the selected target cell population a composition comprising a monomeric cytotoxic fusion protein comprising an N-terminus coupled to a C terminus, wherein the N-terminus comprises diphtheria toxin fragments A and B and the C-terminus comprises human IL-2, wherein at least 95.0% of the total cytotoxic fusion protein content of the composition is a monomeric cytotoxic fusion protein, and wherein said administering is effective to sensitize the target cell population to treatment with a PD-1 pathway inhibitor.

29. The method according to claim 28, wherein the target cells are T-cells.

30. The method according to claim 28, wherein the target cells are tumor-infiltrating T regulatory cells (Tregs) or T effector cells (Teffs).

31. The method according to claim 28, wherein the target cells are CD25⁺ cells.

32. The method according to claim 28, wherein the target cells are cancer cells.

33. The method according to claim 32, wherein the cancer is a lymphoma.

34. The method according to claim 33, wherein the lymphoma is a cutaneous T-cell lymphoma (CTCL) or a peripheral T-cell lymphoma (PTCL).

35. The method according to claim 34, wherein the CTCL is selected from the group consisting of mycosis fungoides (MF), Sézary syndrome (SS), granulomatous slack skin (GSS), lymphomatoid papulosis (LyP), pagetoid reticulosis (PR), primary cutaneous anaplastic large cell lymphomas (PCALCL), and subcutaneous panniculitis T-cell lymphoma (SPTCL).

36. The method according to claim 34, wherein the PTCL is selected from the group consisting of peripheral T-cell lymphoma not otherwise specified (PTCL-NOS), angioimmunoblastic T-cell lymphoma (AITL), systemic anaplastic large cell lymphoma-anaplastic lymphoma kinase positive (sALCL-ALK⁺), systemic anaplastic large cell lymphoma-anaplastic lymphoma kinase negative (sALCL-ALK⁻), adult T-cell leukemia/lymphoma (ATLL), and enteropathy-associated T-cell lymphoma (EATL).

37. The method according to claim 32, wherein the cancer is selected from the group consisting of breast cancer, uterine corpus cancer, cervical cancer, ovarian cancer, prostate cancer, lung cancer, stomach cancer, non-small cell lung cancer, spleen cancer, head and neck squamous cell carcinoma, esophageal cancer, bladder cancer, melanoma, colorectal cancer, kidney cancer, non-Hodgkin lymphoma, urothelial cancer, sarcoma, blood cell carcinoma, bile duct carcinoma, gallbladder carcinoma, thyroid carcinoma, prostate cancer, testicular carcinoma, thymic carcinoma, and hepatocarcinoma.

38. The method according to claim 32, wherein the cancer is not melanoma.

39. The method according to claim 28, wherein the target cell population is a population of human cells.

40. The method according to claim 28, wherein the target cell population exhibits resistance to treatment with PD-1 inhibitor monotherapy prior to said administering.

41. The method according to claim 28, wherein the cytotoxic fusion protein comprises the amino acid sequence of SEQ ID NO:1.

42. The method according to claim 28, wherein the PD-1 pathway inhibitor is an anti-PD-1 antibody selected from the group consisting of nivolumab (OPDIVO®), pembrolizumab (KEYTRUDA®), cemiplimab (LIBTAYO®), pidilizumab (CT-011), REGN2810 (SAR-439684), spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514 (MEDI0680), and PF-06801591.

43. The method according to claim 28, wherein the method is carried out in vitro.

44. The method according to claim 28, wherein the method is carried out in vivo.

45. The method according to claim 28, wherein said selecting comprises selecting a subject having a CD25⁺ lymphoma or a CD25⁺ tumor and said administering is to the selected subject.

46. The method according to claim 45, wherein said administering the cytotoxic fusion protein is carried out at a dose of 6-12 μg/kg/day.

47. The method according to claim 28, wherein said administering the recombinant cytotoxic fusion protein is carried out orally, topically, transdermally, parenterally, intradermally, intrapulmonary, intramuscularly, intraperitoneally, intravenously, intratumorally, subcutaneously, or by intranasal instillation, by intracavitary or intravesical instillation, intraocularly, intraarterially, intralesionally, or by application to mucous membranes.

48. The method according to claim 47 further comprising:
administering the PD-1 pathway inhibitor to the selected cells.

49. The method according to claim 48, wherein said administering the recombinant cytotoxic fusion protein is carried out before, after, or simultaneously with the PD-1 pathway inhibitor.

50. The method according to claim 48 or claim 49, wherein said administering the composition comprising a monomeric cytotoxic fusion protein and said administering the PD-1 pathway inhibitor are effective to increase the proportion of CD8⁺ cells in the target cell population relative to when the target cell population is administered a PD-1 monotherapy.

51. The method according to claim 50, wherein the target cell population is a tumor cell population.

52. A kit comprising:
(i) a monomeric cytotoxic fusion protein comprising an N-terminus coupled to a C-terminus, where the N-terminus comprises diphtheria toxin fragments A and B and the C-terminus comprises human IL-2, and wherein at least 95.0% of the total cytotoxic fusion protein content of the composition is the monomeric cytotoxic fusion protein and
(ii) a programmed cell death-1 receptor (PD-1) pathway inhibitor.


SEQUENCE LISTING (1 sequence)

SEQ ID NO: 1 - protein, 521 aa, Artificial Sequence
  Denileukin diftitox
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT